(19) United States
(12) Patent Application Publication
(10) Pub. No.: US 2026/0060982 A1
(43) Pub. Date: Mar. 5, 2026

(54) USE OF BTK INHIBITOR AND SINGLE-DOSE DRUG

(71) Applicant: HUBEI BIO-PHARMACEUTICAL INDUSTRIAL TECHNOLOGICAL INSTITUTE INC., Wuhan (CN)

(72) Inventors: Jun YANG, Wuhan (CN); Shengji ZHU, Wuhan (CN); Hongna SUN, Wuhan (CN); Jinping LI, Wuhan (CN); Mingwei SU, Wuhan (CN); Jie ZHU, Wuhan (CN); Ling CAI, Wuhan (CN); Zhenyu HE, Wuhan (CN); Sheng CHEN, Wuhan (CN); Qing XU, Wuhan (CN); Xuejun ZHANG, Wuhan (CN); Lie LI, Wuhan (CN)

(21) Appl. No.: 19/382,302

(22) Filed: Nov. 7, 2025

Related U.S. Application Data
(63) Continuation of application No. PCT/CN2024/092670, filed on May 11, 2024.

(30) Foreign Application Priority Data
May 12, 2023 (CN) 202310549229.X

(51) Int. Cl. A61K 31/5025; A61P 17/00; A61P 19/02; A61P 37/06; A61P 37/08

(52) U.S. Cl. CPC A61K 31/5025; A61P 17/00; A61P 19/02; A61P 37/06; A61P 37/08

(57) ABSTRACT

Provided is use of a compound represented by Formula (I), or a stereoisomer, tautomer, solvate or pharmaceutically acceptable salt of the compound represented by Formula (I) in the manufacture of a medicament for treating and/or preventing an immune system disease.

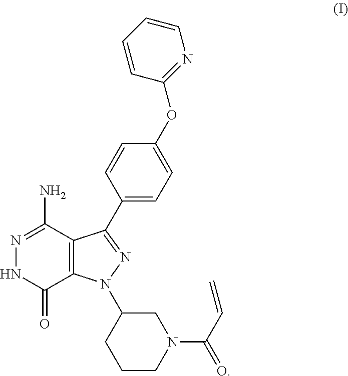 [SMILES: C=CC(=O)N1CCCC(n2nc(-c3ccc(Oc4ccccn4)cc3)c3c(N)n[nH]c(=O)c32)C1]

### CROSS-REFERENCE TO RELATED APPLICATIONS

This application is a continuation of International Patent Application No. PCT/CN2024/092670 filed on May 11, 2024, which claims priority and benefits of the patent application No. 202310549229.X, filed with the China National Intellectual Property Administration on May 12, 2023, which is incorporated herein by reference in its entirety.

### FIELD

The present disclosure belongs to the technical field of biopharmaceuticals. Specifically, the present disclosure relates to use of a BTK inhibitor and a single-dose medicament. More specifically, the present disclosure relates to use of a BTK inhibitor in the manufacture of a medicament for treating and/or preventing an immune system disease and a single-dose medicament.

### BACKGROUND

Bruton's tyrosine kinase (BTK), a member of the Tec family of non-receptor tyrosine kinases, is a key signaling enzyme expressed in all hematopoietic cell types except T lymphocytes and natural killer cells. BTK plays a crucial role in the B-cell signaling pathway that connects cell surface B-cell receptor (BCR) stimulation to downstream intracellular responses. In certain malignancies, BTK is overexpressed in B cells, which is associated with increased proliferation and survival of tumor cells. Inhibition of BTK affects the B-cell signaling pathway, prevents B-cell activation, and inhibits the growth of malignant B cells. Therefore, inhibition of BTK activity can be used to treat cancer, as well as B-cell lymphomas, leukemias, and other hematological malignancies. Many clinical trials have shown that BTK inhibitors are effective in treating cancer. The BTK inhibitor ibrutinib (PCI-32765) has been approved by the U.S. Food and Drug Administration for the treatment of patients with mantle cell lymphoma (MCL) or chronic lymphocytic leukemia (CLL).

The present disclosure aims to provide use of a BTK inhibitor in the manufacture of a medicament for treating and/or preventing an immune system disease and a single-dose medicament.

### SUMMARY

The present disclosure aims to solve, at least to a certain extent, one of the technical problems existing in the prior art. To this end, the present disclosure provides use of a BTK inhibitor in the manufacture of a medicament for treating and/or preventing an immune system disease.

In one aspect of the present disclosure, the present disclosure provides use of a compound represented by Formula (I), or a stereoisomer, tautomer, solvate or pharmaceutically acceptable salt of the compound represented by Formula (I) in the manufacture of a medicament for treating and/or preventing an immune system disease:

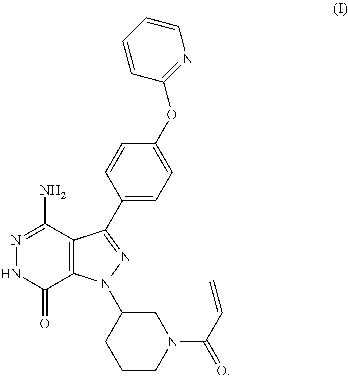 [SMILES: C=CC(=O)N1CCCC(n2nc(-c3ccc(Oc4ccccn4)cc3)c3c(N)n[nH]c(=O)c32)C1]

The above-mentioned compound represented by Formula (I) or the stereoisomer, tautomer, solvate, or pharmaceutically acceptable salt of the compound represented by Formula (I) serves as a highly selective irreversible inhibitor of BTK kinase, which can effectively treat and/or prevent the immune system disease.

In another aspect of the present disclosure, the present disclosure provides use of a compound represented by Formula (I), or a stereoisomer, tautomer, solvate or pharmaceutically acceptable salt of the compound represented by Formula (I) in the treatment and/or prevention of an immune system disease:

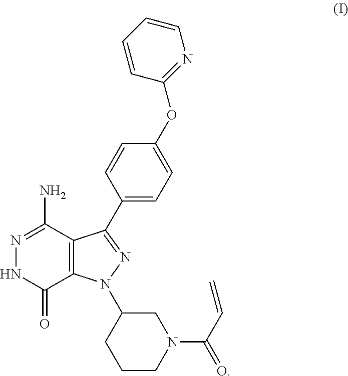 [SMILES: C=CC(=O)N1CCCC(n2nc(-c3ccc(Oc4ccccn4)cc3)c3c(N)n[nH]c(=O)c32)C1]

The above-mentioned compound represented by Formula (I) or the stereoisomer, tautomer, solvate, or pharmaceutically acceptable salt of the compound represented by Formula (I) serves as a highly selective irreversible inhibitor of BTK kinase, which can effectively treat and/or prevent the immune system disease.

In another aspect of the present disclosure, the present disclosure provides a compound represented by Formula (I), or a stereoisomer, tautomer, solvate or pharmaceutically acceptable salt of the compound represented by Formula (I) for use in treating and/or preventing an immune system disease:

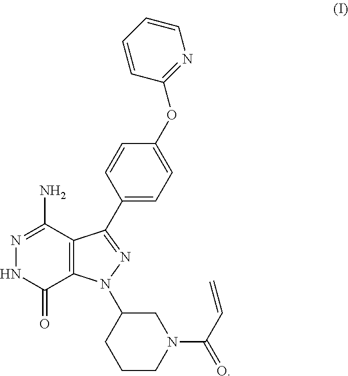 [SMILES: C=CC(=O)N1CCCC(n2nc(-c3ccc(Oc4ccccn4)cc3)c3c(N)n[nH]c(=O)c32)C1]

The above-mentioned compound represented by Formula (I) or the stereoisomer, tautomer, solvate, or pharmaceutically acceptable salt of the compound represented by Formula (I) serves as a highly selective irreversible inhibitor of BTK kinase, which can effectively treat and/or prevent the immune system disease.

According to an embodiment of the present disclosure, the above three aspects of use may further include at least one of the following technical features:

According to an embodiment of the present disclosure, the immune system disease includes an autoimmune disease and/or a skin allergy disorder.

According to an embodiment of the present disclosure, the immune system disease includes rheumatoid arthritis, urticaria, skin inflammation, and/or eczema.

According to an embodiment of the present disclosure, an effective dose of the compound represented by Formula (I) or the stereoisomer, tautomer, solvate, or pharmaceutically acceptable salt of the compound represented by Formula (I) ranges from about 5 mg to 800 mg.

According to an embodiment of the present disclosure, an effective dose of the compound represented by Formula (I) or the stereoisomer, tautomer, solvate, or pharmaceutically acceptable salt of the compound represented by Formula (I) ranges from about 10 mg to 800 mg.

According to an embodiment of the present disclosure, an effective dose of the compound represented by Formula (I) or the stereoisomer, tautomer, solvate, or pharmaceutically acceptable salt of the compound represented by Formula (I) ranges from about 20 mg to 800 mg.

According to an embodiment of the present disclosure, an effective dose of the compound represented by Formula (I) or the stereoisomer, tautomer, solvate, or pharmaceutically acceptable salt of the compound represented by Formula (I) ranges from about 25 mg to 800 mg.

In another aspect of the present disclosure, the present disclosure provides a single-dose medicament, which includes, as an active ingredient, about 1 mg to 800 mg of a compound represented by Formula (I), or a stereoisomer, tautomer, solvate or pharmaceutically acceptable salt of the compound represented by Formula (I):

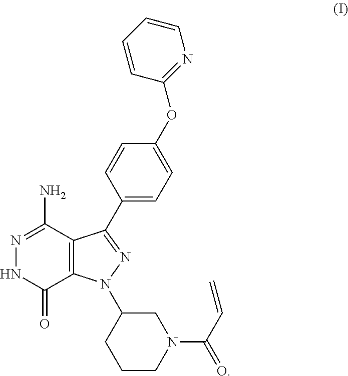 [SMILES: C=CC(=O)N1CCCC(n2nc(-c3ccc(Oc4ccccn4)cc3)c3c(N)n[nH]c(=O)c32)C1]

According to an embodiment of the present disclosure, the single-dose medicament can effectively treat and/or prevent the immune system disease.

In another aspect of the present disclosure, the present disclosure provides a single-dose medicament for use in treating and/or preventing an immune system disease. The single-dose medicament includes, as an active ingredient, about 1 mg to 800 mg of a compound represented by Formula (I), or a stereoisomer, tautomer, solvate or pharmaceutically acceptable salt of the compound represented by Formula (I):

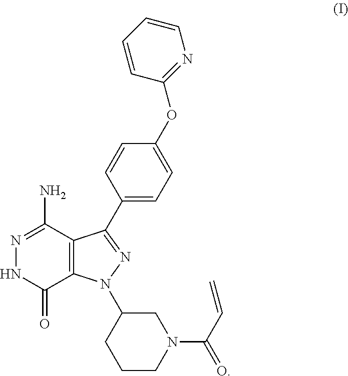 [SMILES: C=CC(=O)N1CCCC(n2nc(-c3ccc(Oc4ccccn4)cc3)c3c(N)n[nH]c(=O)c32)C1]

According to an embodiment of the present disclosure, the single-dose medicament according to the above two aspects may further include at least one of the following technical features:

According to an embodiment of the present disclosure, the single-dose medicament includes, as an active ingredient, about 5 mg to 800 mg of the compound represented by Formula (I) or the stereoisomer, tautomer, solvate, or pharmaceutically acceptable salt of the compound represented by Formula (I).

According to an embodiment of the present disclosure, the single-dose medicament includes, as an active ingredient, about 10 mg to 800 mg of the compound represented by Formula (I) or the stereoisomer, tautomer, solvate, or pharmaceutically acceptable salt of the compound represented by Formula (I).

According to an embodiment of the present disclosure, the single-dose medicament includes, as an active ingredient, about 20 mg to 800 mg of the compound represented by Formula (I) or the stereoisomer, tautomer, solvate, or pharmaceutically acceptable salt of the compound represented by Formula (I).

According to an embodiment of the present disclosure, the single-dose medicament includes, as an active ingredient, about 25 mg to 800 mg of the compound represented by Formula (I) or the stereoisomer, tautomer, solvate, or pharmaceutically acceptable salt of the compound represented by Formula (I).

According to an embodiment of the present disclosure, the immune system disease includes rheumatoid arthritis and/or a skin allergy disorder.

According to an embodiment of the present disclosure, the immune system disease includes rheumatoid arthritis, urticaria, skin inflammation, and/or eczema.

According to an embodiment of the present disclosure, the urticaria includes chronic spontaneous urticaria.

In another aspect of the present disclosure, the present disclosure provides a method for treating and/or preventing an immune system disease. The method includes administering to a subject a pharmaceutically acceptable dose of a compound represented by Formula (I), or a stereoisomer, tautomer, solvate or pharmaceutically acceptable salt of the compound represented by Formula (I), or the above-mentioned single-dose medicament:

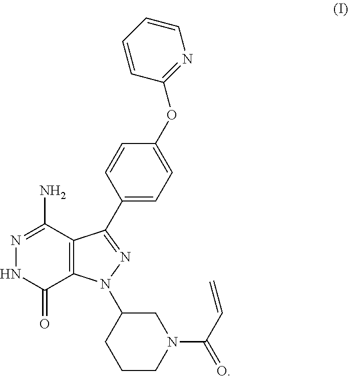 [SMILES: C=CC(=O)N1CCCC(n2nc(-c3ccc(Oc4ccccn4)cc3)c3c(N)n[nH]c(=O)c32)C1]

In some optional embodiments of the present disclosure, the immune system disease includes an autoimmune disease and/or a skin allergy disorder.

In some optional embodiments of the present disclosure, the immune system disease includes rheumatoid arthritis, urticaria, skin inflammation, and/or eczema.

Additional aspects and advantages of the present disclosure will be provided in part in the following description, or will become apparent in part from the following description, or can be learned from practicing of the present disclosure.

### BRIEF DESCRIPTION OF THE DRAWINGS

The foregoing and/or additional aspects and advantages of the present disclosure will become apparent and readily appreciated from the following description of the embodiments in conjunction with the accompanying drawings.

FIG. 1 shows the correlation between the Cₘₐₓ of compound (I) capsule and the dose of compound (I) after a single administration in each dose group in the clinical trial of Example 2 of the present disclosure.

FIG. 2 shows the correlation between the AUC₀₋ₜ of compound (I) capsule and the dose of compound (I) after a single administration in each dose group (10 mg to 100 mg) in the clinical trial of Example 2 of the present disclosure.

FIG. 3 shows the correlation between the AUC₀₋ₜ of compound (I) capsule and the dose of compound (I) after a single administration in each dose group (200 mg to 800 mg) in the clinical trial of Example 2 of the present disclosure.

FIG. 4 shows the changes in BTK target occupancy rate over time in each dose group of compound (I) capsule in the clinical trial of Example 2 of the present disclosure.

FIG. 5 shows a curve fitting of Cₘₐₓ and maximum BTK target occupancy rate in all subjects in the clinical trial of Example 2 of the present disclosure.

FIG. 6 shows a curve fitting of AUCₗₐₛₜ and maximum BTK target occupancy rate in all subjects in the clinical trial of Example 2 of the present disclosure.

### DETAILED DESCRIPTION

The embodiments of the present disclosure are described in detail below. The embodiments described below are exemplary and are only used to explain the present disclosure, and are not to be construed as limiting the present disclosure.

It should be noted that the terms “first” and “second” are used for descriptive purposes only and are not to be construed as indicating or implying relative importance or implicitly indicating the number of technical features indicated. Thus, a feature defined as “first” or “second” may explicitly or implicitly include one or more of such features. Further, in the description of the present disclosure, “plurality” or “more” means two or more unless otherwise specified.

The endpoints of the ranges and any values disclosed herein shall not limited to the exact range or value, and those ranges or values should be understood to include values close to those ranges or values. For numerical ranges, endpoints of the respective ranges, an endpoint of respective ranges and an individual point value, and individual point values may be combined with each other to obtain one or more new numerical ranges, which should be deemed to be specifically disclosed herein. Certain ranges are provided herein with values preceded by the term “about”. The term “about” is used herein to provide literal support for the exact value that it precedes, as well as a value that is near to or approximately preceded by the term. In determining whether a value is near or approximately a specifically stated value, the near or approximate unstated value may be a value that, in the context in which it is provided, provides a substantial equivalent to the specifically stated value. The term “about” or “approximately” refers to an acceptable error for the particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range.

As used herein, the terms “include” or “comprise” are open expressions, that is, including the contents specified in the present disclosure, but not excluding other contents.

As used herein, the compounds of the present disclosure also include isotopically labeled compounds of the present disclosure, which are identical to those described herein except for the fact that one or more atoms are replaced with an atom having an atomic mass or mass number different from the naturally occurring atomic mass or mass number. Exemplary isotopes that may also be incorporated into the compounds of the present disclosure include isotopes of hydrogen, carbon, nitrogen, oxygen, phosphorus, sulfur, fluorine, and chlorine, such as ²H, ³H, ¹³C, ¹⁴C, ¹⁵N, ¹⁶O, ¹⁷O, ³¹P, ³²P, ³⁶S, ¹⁸F, and ³⁷Cl.

The term “stereoisomer” refers to isomers resulting from different spatial arrangements of atoms in a molecule, including cis-trans isomers, enantiomers, diastereomers, and conformational isomers. The stereochemical definitions and conventions used herein are generally defined according to S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994.

As used herein, the term “tautomer” or “tautomeric form” refers to structural isomers of different energies that are interconvertible via a low energy barrier. If tautomerism is possible (such as in solution), a chemical equilibrium of the tautomers may be achieved. For example, proton tautomers (also known as prototropic tautomers) include interconversions via migration of a proton, such as keto-enol and imine-enamine isomerizations. Valence tautomers include interconversions by reorganization of some of the bonding electrons. Unless otherwise indicated, all tautomeric forms of the compounds of the present disclosure are within the scope of the present disclosure.

As used herein, the term “solvate” refers to an association between one or more solvent molecules and a compound of the present disclosure. Solvents that form solvates include, but are not limited to, water, isopropanol, ethanol, methanol, dimethyl sulfoxide, ethyl acetate, acetic acid, and aminoethanol. The term “hydrate” refers to an association in which the solvent molecule is water.

As used herein, the term “pharmaceutically acceptable” means that the substance or composition must be chemically and/or toxicologically compatible with the other ingredients including the formulation and/or the mammal to be treated therewith.

As used herein, the term “pharmaceutically acceptable salt” refers to both organic and inorganic salts of the compounds of the present disclosure. Pharmaceutically acceptable salts are well known in the art, as described in SM Berge et al., describe pharmaceutically acceptable salts in detail in J. Pharmaceutical Sciences, 1977, 66: 1-19.

As used herein, the term “treatment” or “treating” refers to administering a medicament for the purpose of obtaining a desired pharmacological and/or physiological effect. The effect may be prophylactic in terms of complete or partial prevention of the disease or its symptoms, and/or therapeutic in terms of partial or complete cure of the disease and/or its adverse effects. As used herein, “treatment” or “treating” encompasses diseases in mammals, particularly humans, and includes: (a) preventing the occurrence of a disease or condition in an individual who is susceptible to the disease but has not yet been diagnosed with the disease; (b) inhibiting the disease, such as arresting the progression of the disease; or (c) alleviating the disease, such as alleviating the symptoms associated with the disease. As used herein, “treatment” or “treating” encompasses any administration of a medicament or compound to an individual to treat, cure, alleviate, ameliorate, mitigate, or inhibit a disease in the individual, including but not limited to administering a medicament containing a compound described herein to an individual in need thereof.

The present disclosure provides use of a BTK inhibitor in the manufacture of a medicament for treating and/or preventing an immune system disease and a single-dose medicament, each of which will be described in detail below.

### Use

In one aspect of the present disclosure, the present disclosure provides use of a compound represented by Formula (I), or a stereoisomer, tautomer, solvate or pharmaceutically acceptable salt of the compound represented by Formula (I) in the manufacture of a medicament for treating and/or preventing an immune system disease:

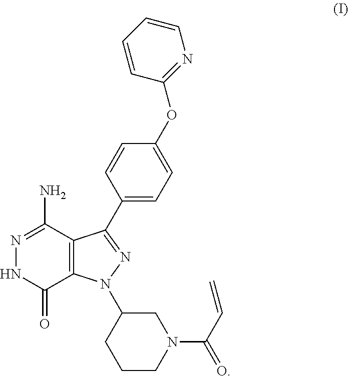 [SMILES: C=CC(=O)N1CCCC(n2nc(-c3ccc(Oc4ccccn4)cc3)c3c(N)n[nH]c(=O)c32)C1]

The above-mentioned compound represented by Formula (I) or the stereoisomer, tautomer, solvate, or pharmaceutically acceptable salt of the compound represented by Formula (I) (hereinafter referred to as the compound) according to the embodiments of the present disclosure serves as a highly selective irreversible inhibitor of BTK kinase, which can effectively treat and/or prevent the immune system disease. Moreover, the above-mentioned compound has a large medicament safety window and is characterized by low effective dose and better safety in clinical applications, thereby greatly improving the patient compliance.

According to the embodiments of the present disclosure, the above-mentioned compound has the advantages of high enzyme activity, good water solubility, low human plasma protein binding rate, high oral bioavailability, or long oral half-life. It has better safety, tolerability, pharmacokinetics, pharmacodynamics, and effectiveness in clinical applications, thereby greatly improving the patient compliance.

In another aspect of the present disclosure, the present disclosure provides use of a compound represented by Formula (I), or a stereoisomer, tautomer, solvate or pharmaceutically acceptable salt of the compound represented by Formula (I) in the treatment and/or prevention of an immune system disease:

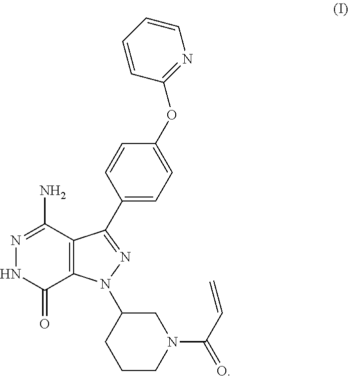 [SMILES: C=CC(=O)N1CCCC(n2nc(-c3ccc(Oc4ccccn4)cc3)c3c(N)n[nH]c(=O)c32)C1]

The above-mentioned compound represented by Formula (I) or the stereoisomer, tautomer, solvate, or pharmaceutically acceptable salt of the compound represented by Formula (I) serves as a highly selective irreversible inhibitor of BTK kinase, has the advantages of high enzyme activity, good water solubility, low human plasma protein binding rate, high oral bioavailability, or long oral half-life, and can effectively treat and/or prevent the immune system disease.

In another aspect of the present disclosure, the present disclosure provides a compound represented by Formula (I), or a stereoisomer, tautomer, solvate or pharmaceutically acceptable salt of the compound represented by Formula (I) for use in treating and/or preventing an immune system disease:

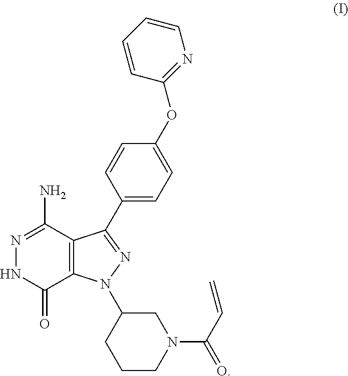 [SMILES: C=CC(=O)N1CCCC(n2nc(-c3ccc(Oc4ccccn4)cc3)c3c(N)n[nH]c(=O)c32)C1]

The above-mentioned compound represented by Formula (I) or the stereoisomer, tautomer, solvate, or pharmaceutically acceptable salt of the compound represented by Formula (I) serves as a highly selective irreversible inhibitor of BTK kinase, has the advantages of high enzyme activity, good water solubility, low human plasma protein binding rate, high oral bioavailability, or long oral half-life, and can effectively treat and/or prevent the immune system disease.

According to an embodiment of the present disclosure, the above three aspects of use may further include at least one of the following technical features:

In some optional embodiments of the present disclosure, the immune system disease includes an autoimmune disease and/or a skin allergy disorder. The compound according to the embodiments of the present disclosure has high target selectivity and exhibits significant efficacy in two different animal models.

As used herein, “skin allergy disorder” and “skin allergy reaction” are synonymous unless otherwise specified.

In some optional embodiments of the present disclosure, the immune system disease includes rheumatoid arthritis, urticaria, skin inflammation, and/or eczema.

In some optional embodiments of the present disclosure, the autoimmune disease includes rheumatoid arthritis and/or urticaria.

In some optional embodiments of the present disclosure, the skin allergy disorder includes urticaria, skin inflammation, and/or eczema.

In some optional embodiments of the present disclosure, the urticaria includes chronic spontaneous urticaria.

According to an embodiment of the present disclosure, an effective dose of the compound represented by Formula (I) or the stereoisomer, tautomer, solvate, or pharmaceutically acceptable salt of the compound represented by Formula (I) ranges from about 5 mg to 800 mg.

According to an embodiment of the present disclosure, an effective dose of the compound represented by Formula (I) or the stereoisomer, tautomer, solvate, or pharmaceutically acceptable salt of the compound represented by Formula (I) ranges from about 10 mg to 800 mg.

According to an embodiment of the present disclosure, an effective dose of the compound represented by Formula (I) or the stereoisomer, tautomer, solvate, or pharmaceutically acceptable salt of the compound represented by Formula (I) ranges from about 20 mg to 800 mg.

According to an embodiment of the present disclosure, an effective dose of the compound represented by Formula (I) or the stereoisomer, tautomer, solvate, or pharmaceutically acceptable salt of the compound represented by Formula (I) ranges from about 25 mg to 800 mg.

According to an embodiment of the present disclosure, an effective dose of the compound represented by Formula (I) or the stereoisomer, tautomer, solvate, or pharmaceutically acceptable salt of the compound represented by Formula (I) ranges from about 10 mg to 100 mg.

According to an embodiment of the present disclosure, an effective dose of the compound represented by Formula (I) or the stereoisomer, tautomer, solvate, or pharmaceutically acceptable salt of the compound represented by Formula (I) ranges from about 200 mg to 800 mg.

According to an embodiment of the present disclosure, an effective dose of the compound represented by Formula (I) or the stereoisomer, tautomer, solvate, or pharmaceutically acceptable salt of the compound represented by Formula (I) ranges from about 1 mg to 5 mg, 1 mg to 10 mg, 1 mg to 25 mg, 1 mg to 50 mg, 1 mg to 100 mg, 1 mg to 200 mg, 1 mg to 400 mg, 1 mg to 600 mg, 1 mg to 800 mg, 5 mg to 10 mg, 5 mg to 25 mg, 5 mg to 50 mg, 5 mg to 100 mg, 5 mg to 200 mg, 5 mg to 400 mg, 5 mg to 600 mg, 5 mg to 800 mg, 10 mg to 25 mg, 10 mg to 50 mg, 10 mg to 100 mg, 10 mg to 200 mg, 10 mg to 400 mg, 10 mg to 600 mg, 10 mg to 800 mg, 25 mg to 50 mg, 25 mg to 100 mg, 25 mg to 200 mg, 25 mg to 400 mg, 25 mg to 600 mg, 25 mg to 800 mg, 50 mg to 100 mg, 50 mg to 200 mg, 50 mg to 400 mg, 50 mg to 600 mg, 50 mg to 800 mg, 100 mg to 200 mg, 100 mg to 400 mg, 100 mg to 600 mg, 100 mg to 800 mg, 200 mg to 400 mg, 200 mg to 600 mg, 200 mg to 800 mg, 400 mg to 600 mg, 400 mg to 800 mg, or 600 mg to 800 mg.

In some optional embodiments of the present disclosure, the type of the medicament is not specifically limited, as long as it includes the compound represented by Formula (I) or the stereoisomer, tautomer, solvate, or pharmaceutically acceptable salt of the compound represented by Formula (I). In some optional embodiments of the present disclosure, the medicament is suitable for oral administration. For example, it can be a solid oral formulation, which includes the compound represented by Formula (I) or the stereoisomer, tautomer, solvate, or pharmaceutically acceptable salt of the compound represented by Formula (I). Preferably, the compound represented by Formula (I) or the stereoisomer, tautomer, solvate, or pharmaceutically acceptable salt of the compound represented by Formula (I) in the solid oral formulation serves as the only active ingredient. In an optional embodiment of the present disclosure, the solid oral formulation includes but is not limited to capsules, tablets, dispersible powders, or granules.

In an optional embodiment of the present disclosure, the solid oral formulation may further include excipients, which are well known to those skilled in the art. In an optional embodiment of the present disclosure, the excipient includes but is not limited to at least one of a filler, a diluent, a disintegrant, a lubricant, a surfactant, or a binder. In some optional embodiments of the present disclosure, the filler includes but is not limited to at least one of pregelatinized starch, mannitol, microcrystalline cellulose, or lactose. In some optional embodiments of the present disclosure, the diluent includes but is not limited to at least one of calcium carbonate, sodium carbonate, lactose, calcium phosphate, or sodium phosphate. In some optional embodiments of the present disclosure, the disintegrant includes but is not limited to at least one of cross-linked sodium carboxymethyl cellulose, sodium starch glycolate, or cross-linked polyvinylpyrrolidone. In some optional embodiments of the present disclosure, the lubricant includes but is not limited to at least one of magnesium stearate, calcium stearate, or talc. In some optional embodiments of the present disclosure, the binder includes but is not limited to at least one of hypromellose, povidone K30, or hydroxypropyl cellulose. In some optional embodiments of the present disclosure, the surfactant includes but is not limited to at least one of sodium dodecyl sulfate or Tween 80.

It should be noted that the efficacy of the medicament is mainly determined by the active ingredient, i.e., the compound represented by Formula (I) or the stereoisomer, tautomer, solvate, or pharmaceutically acceptable salt of the compound represented by Formula (I).

In some optional embodiments of the present disclosure, the solid oral formulation includes but is not limited to capsules, in which the compound represented by Formula (I) or the stereoisomer, tautomer, solvate, or pharmaceutically acceptable salt of the compound represented by Formula (I) serves as the only active ingredient.

In some optional embodiments of the present disclosure, the excipients of the capsule include but are not limited to at least one of a filler, a disintegrant, a lubricant, a surfactant, or a binder.

In some optional embodiments of the present disclosure, the capsule includes the compound represented by Formula (I) or the stereoisomer, tautomer, solvate, or pharmaceutically acceptable salt of the compound represented by Formula (I), pregelatinized starch, cross-linked sodium carboxymethyl cellulose, and magnesium stearate. The above-mentioned capsule can be used for subsequent clinical trials, such as trials on efficacy, pharmacokinetics, safety, and the like.

### Single-Dose Medicament

In another aspect of the present disclosure, the present disclosure provides a single-dose medicament, which includes, as an active ingredient, about 1 mg to 800 mg of a compound represented by Formula (I), or a stereoisomer, tautomer, solvate or pharmaceutically acceptable salt of the compound represented by Formula (I):

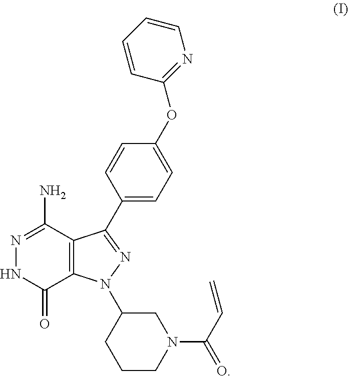 [SMILES: C=CC(=O)N1CCCC(n2nc(-c3ccc(Oc4ccccn4)cc3)c3c(N)n[nH]c(=O)c32)C1]

In some optional embodiments of the present disclosure, the present disclosure provides a single-dose medicament for treating and/or preventing an immune system disease, which includes, as an active ingredient, about 1 mg to 800 mg of a compound represented by Formula (I), or a stereoisomer, tautomer, solvate or pharmaceutically acceptable salt of the compound represented by Formula (I):

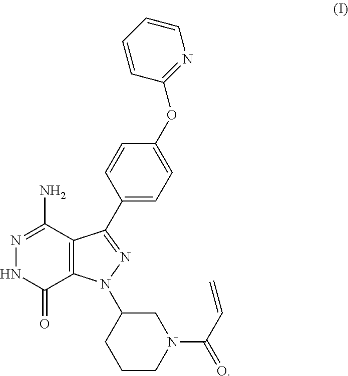 [SMILES: C=CC(=O)N1CCCC(n2nc(-c3ccc(Oc4ccccn4)cc3)c3c(N)n[nH]c(=O)c32)C1]

The above-mentioned compound represented by Formula (I) or the stereoisomer, tautomer, solvate, or pharmaceutically acceptable salt of the compound represented by Formula (I) serves as a highly selective irreversible inhibitor of BTK kinase, which can effectively treat and/or prevent the immune system disease.

As used herein, the term “single-dose” refers to a dose for a single use. Exemplarily, the “single-dose medicament” refers to a dose that can achieve the effect of treating and/or preventing the immune system disease by a single addition of the compound represented by Formula (I) or the stereoisomer, tautomer, solvate, or pharmaceutically acceptable salt of the compound represented by Formula (I). Preferably, in the single-dose medicament, the compound represented by Formula (I) or the stereoisomer, tautomer, solvate, or pharmaceutically acceptable salt of the compound represented by Formula (I) serves as the sole active ingredient.

In some optional embodiments of the present disclosure, the single-dose medicament includes, as an active ingredient, about 1 mg to 800 mg of the compound represented by Formula (I) or the stereoisomer, tautomer, solvate, or pharmaceutically acceptable salt of the compound represented by Formula (I).

In some optional embodiments of the present disclosure, the single-dose medicament includes, as an active ingredient, about 1 mg, 5 mg, 10 mg, 25 mg, 50 mg, 100 mg, 200 mg, 400 mg, 600 mg, or 800 mg, or a range value between any two numbers of the compound represented by Formula (I) or the stereoisomer, tautomer, solvate, or pharmaceutically acceptable salt of the compound represented by Formula (I), for example: 1 mg to 5 mg, 1 mg to 10 mg, 1 mg to 25 mg, 1 mg to 50 mg, 1 mg to 100 mg, 1 mg to 200 mg, 1 mg to 400 mg, 1 mg to 600 mg, 1 mg to 800 mg, 5 mg to 10 mg, 5 mg to 25 mg, 5 mg to 50 mg, 5 mg to 100 mg, 5 mg to 200 mg, 5 mg to 400 mg, 5 mg to 600 mg, 5 mg to 800 mg, 10 mg to 25 mg, 10 mg to 50 mg, 10 mg to 100 mg, 10 mg to 200 mg, 10 mg to 400 mg, 10 mg to 600 mg, 10 mg to 800 mg, 25 mg to 50 mg, 25 mg to 100 mg, 25 mg to 200 mg, 25 mg to 400 mg, 25 mg to 600 mg, 25 mg to 800 mg, 50 mg to 100 mg, 50 mg to 200 mg, 50 mg to 400 mg, 50 mg to 600 mg, 50 mg to 800 mg, 100 mg to 200 mg, 100 mg to 400 mg, 100 mg to 600 mg, 100 mg to 800 mg, 200 mg to 400 mg, 200 mg to 600 mg, 200 mg to 800 mg, 400 mg to 600 mg, 400 mg to 800 mg, or 600 mg to 800 mg.

According to an embodiment of the present disclosure, the single-dose medicament includes, as an active ingredient, about 5 mg to 800 mg of the compound represented by Formula (I) or the stereoisomer, tautomer, solvate, or pharmaceutically acceptable salt of the compound represented by Formula (I).

According to an embodiment of the present disclosure, the single-dose medicament includes, as an active ingredient, about 10 mg to 800 mg of the compound represented by Formula (I) or the stereoisomer, tautomer, solvate, or pharmaceutically acceptable salt of the compound represented by Formula (I).

In some optional embodiments of the present disclosure, the single-dose medicament includes, as an active ingredient, about 20 mg to 800 mg of the compound represented by Formula (I) or the stereoisomer, tautomer, solvate, or pharmaceutically acceptable salt of the compound represented by Formula (I). In this way, the maximum BTK target occupancy rate of the single-dose medicament can be further increased, and the occupancy rate can reach up to 100%.

In some optional embodiments of the present disclosure, the single-dose medicament includes, as an active ingredient, about 25 mg to 800 mg of the compound represented by Formula (I) or the stereoisomer, tautomer, solvate, or pharmaceutically acceptable salt of the compound represented by Formula (I).

According to an embodiment of the present disclosure, the single-dose medicament includes, as an active ingredient, about 10 mg to 100 mg of the compound represented by Formula (I) or the stereoisomer, tautomer, solvate, or pharmaceutically acceptable salt of the compound represented by Formula (I).

According to an embodiment of the present disclosure, the single-dose medicament includes, as an active ingredient, about 200 mg to 800 mg of the compound represented by Formula (I) or the stereoisomer, tautomer, solvate, or pharmaceutically acceptable salt of the compound represented by Formula (I).

In some optional embodiments of the present disclosure, the immune system disease includes an autoimmune disease and/or a skin allergy disorder.

In some optional embodiments of the present disclosure, the immune system disease includes rheumatoid arthritis, urticaria, skin inflammation, and/or eczema.

In some optional embodiments of the present disclosure, the immune disease includes rheumatoid arthritis and/or a skin allergy disorder.

In some optional embodiments of the present disclosure, the autoimmune disease includes rheumatoid arthritis and/or urticaria.

In some optional embodiments of the present disclosure, the skin allergy disorder includes urticaria, skin inflammation, and/or eczema.

In some optional embodiments of the present disclosure, the skin allergy disorder includes urticaria or eczema.

In some optional embodiments of the present disclosure, the urticaria includes chronic spontaneous urticaria.

Method for Treating and/or Preventing an Immune System Disease

In another aspect of the present disclosure, the present disclosure provides a method for treating and/or preventing an immune system disease. The method includes administering to a subject a pharmaceutically acceptable dose of a compound represented by Formula (I), or a stereoisomer, tautomer, solvate or pharmaceutically acceptable salt of the compound represented by Formula (I), or the above-mentioned single-dose medicament:

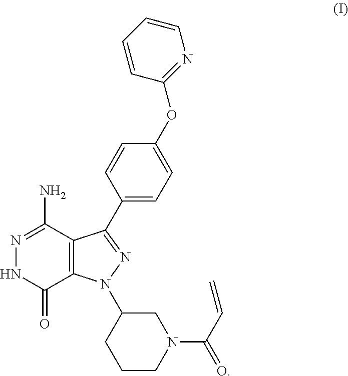 [SMILES: C=CC(=O)N1CCCC(n2nc(-c3ccc(Oc4ccccn4)cc3)c3c(N)n[nH]c(=O)c32)C1]

In some optional embodiments of the present disclosure, the immune system disease includes an autoimmune disease and/or a skin allergy disorder.

In some optional embodiments of the present disclosure, the immune system disease includes rheumatoid arthritis, urticaria, skin inflammation, and/or eczema.

In some optional embodiments of the present disclosure, the autoimmune disease includes rheumatoid arthritis and/or urticaria.

In some optional embodiments of the present disclosure, the skin allergy disorder includes urticaria, skin inflammation, and/or eczema.

In some optional embodiments of the present disclosure, the urticaria includes chronic spontaneous urticaria.

According to an embodiment of the present disclosure, an effective dose of the compound represented by Formula (I) or the stereoisomer, tautomer, solvate, or pharmaceutically acceptable salt of the compound represented by Formula (I) ranges from about 5 mg to 800 mg.

According to an embodiment of the present disclosure, an effective dose of the compound represented by Formula (I) or the stereoisomer, tautomer, solvate, or pharmaceutically acceptable salt of the compound represented by Formula (I) ranges from about 10 mg to 800 mg.

According to an embodiment of the present disclosure, an effective dose of the compound represented by Formula (I) or the stereoisomer, tautomer, solvate, or pharmaceutically acceptable salt of the compound represented by Formula (I) ranges from about 20 mg to 800 mg.

According to an embodiment of the present disclosure, an effective dose of the compound represented by Formula (I) or the stereoisomer, tautomer, solvate, or pharmaceutically acceptable salt of the compound represented by Formula (I) ranges from about 25 mg to 800 mg.

According to an embodiment of the present disclosure, an effective dose of the compound represented by Formula (I) or the stereoisomer, tautomer, solvate, or pharmaceutically acceptable salt of the compound represented by Formula (I) ranges from about 10 mg to 100 mg.

According to an embodiment of the present disclosure, an effective dose of the compound represented by Formula (I) or the stereoisomer, tautomer, solvate, or pharmaceutically acceptable salt of the compound represented by Formula (I) ranges from about 200 mg to 800 mg.

According to an embodiment of the present disclosure, an effective dose of the compound represented by Formula (I) or the stereoisomer, tautomer, solvate, or pharmaceutically acceptable salt of the compound represented by Formula (I) ranges from about 1 mg to 5 mg, 1 mg to 10 mg, 1 mg to 25 mg, 1 mg to 50 mg, 1 mg to 100 mg, 1 mg to 200 mg, 1 mg to 400 mg, 1 mg to 600 mg, 1 mg to 800 mg, 5 mg to 10 mg, 5 mg to 25 mg, 5 mg to 50 mg, 5 mg to 100 mg, 5 mg to 200 mg, 5 mg to 400 mg, 5 mg to 600 mg, 5 mg to 800 mg, 10 mg to 25 mg, 10 mg to 50 mg, 10 mg to 100 mg, 10 mg to 200 mg, 10 mg to 400 mg, 10 mg to 600 mg, 10 mg to 800 mg, 25 mg to 50 mg, 25 mg to 100 mg, 25 mg to 200 mg, 25 mg to 400 mg, 25 mg to 600 mg, 25 mg to 800 mg, 50 mg to 100 mg, 50 mg to 200 mg, 50 mg to 400 mg, 50 mg to 600 mg, 50 mg to 800 mg, 100 mg to 200 mg, 100 mg to 400 mg, 100 mg to 600 mg, 100 mg to 800 mg, 200 mg to 400 mg, 200 mg to 600 mg, 200 mg to 800 mg, 400 mg to 600 mg, 400 mg to 800 mg, or 600 mg to 800 mg.

The solutions of the present disclosure will be explained below with reference to the examples. Those skilled in the art will understand that the following examples are only used to illustrate the present disclosure and should not be considered to limit the scope of the present disclosure. Where specific techniques or conditions are not specified in the examples, they are performed according to techniques or conditions described in the literature in the art or according to the product description. The reagents or instruments used are conventional products that can be obtained commercially without indicating the manufacturer.

The preparation of the compound represented by Formula (I) ((R)-1-(1-acryloylpiperidin-3-yl)-4-amino-3-(4-(pyridin-2-yloxy)phenyl)-1H-pyrazolo[3,4-d]pyridazin-7(6H)-one) employed in the examples of the present disclosure is described in Scheme 29 and Example 32 of CN105837576A:

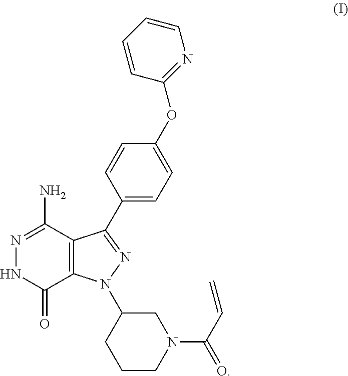 [SMILES: C=CC(=O)N1CCCC(n2nc(-c3ccc(Oc4ccccn4)cc3)c3c(N)n[nH]c(=O)c32)C1]

The CC-292 compound in the examples of the present disclosure is a commercially available product.

### Example 1: Pharmacological Studies of Compound (I)

### 1. In Vitro Efficacy Study of Compound (I)

### 1.1 Study on the Inhibition of BTK Kinase Activity

In this study, compound (I) was prepared by dissolving 1.87 mg of the powder of the compound represented by Formula (I) (purity: 98.7%) in 409 μL of 100% DMSO solution to obtain a 10 mM test compound solution as compound (I).

In this study, CC-292 solution was prepared by dissolving 1.80 mg of CC-292 compound powder (98.7% purity) in 425 μL of 100% DMSO solution to obtain a 10 mM test compound solution as CC-292 solution, subsequently referred to as CC-292.

The inhibitory effects of the test compound (i.e., compound (I)) and the reference medicament (i.e., CC-292) on the BTK kinase activity were examined using Cisbio's homogeneous time-resolved fluorescence conjugate energy transfer (HTRF® method). The specific experimental steps were as follows:

A. 2.5 μL of the diluted compound (compound (I) or CC-292) was taken using Agilent's Bravo, transferred to an assay plate, and centrifuged at 1000 rpm for 1 min.

B. A BTK kinase/peptide solution (2866 μL 1.33× kinase reaction buffer (1330 μL of HTRF® 5× kinase reaction buffer, 13.3 μL of 1 M MgCl₂, 6.65 μL of 1 M DTT, and 3670 μL of ddH₂O), 1.53 μL of 3.92 μM BTK, 12 μL of 500 μM TK substrate, and 120 μL of 2.5 μM SEB) was prepared. The BTK kinase/peptide mix solution was added to the assay plate at 5 μL per well, and centrifuged at 1000 rpm for 1 min. The assay plate was incubated at 23° C. for 15 min.

C. An ATP solution (3968 μL of 1.33× kinase reaction buffer and 32 mL of 10 mM ATP) was prepared, added to the assay plate at 2.5 μL per well, and centrifuged at 1000 rpm for 1 min. The assay plate was incubated at 23° C. for 90 min.

D. An assay reagent (20738 μL of HTRF® Assay Buffer, 157.5 μL of 16.67 M streptavidin-labeled XL665, and 105 μL of 2.67 M anti-TK phospho-peptide substrate antibody) was prepared, added to the assay plate at 10 μL per well, and centrifuged at 1000 rpm for 1 min. The assay plate was incubated at 23° C. for 60 min and read on the Envision multilabel reader.

E. IC₅₀ results were analyzed using XLFIT5 (Formula 205) from IDBS.

The results showed that compound (I) (IC₅₀≤4 nM) had better inhibitory activity against BTK kinase than CC-292 (IC₅₀ approximately 20 nM).

### 1.2 Study on the Antagonism of IgM-Induced Calcium Signaling in RAMOS Cells

In this study, compound (I) was prepared by dissolving 2.63 mg of the powder of the compound represented by Formula (I) (purity: 98.7%) in 575 μL of 100% DMSO solution to obtain a 10 mM test compound solution as compound (I).

In this study, CC-292 solution was prepared by dissolving 2.14 mg of commercially available CC-292 compound powder (98.7% purity) in 505 μL of 100% DMSO to obtain a 10 mM test compound solution as CC-292 solution, subsequently referred to as CC-292.

The inhibitory effects of compound (I) and CC-292 on IgM-induced calcium signaling in RAMOS cells were investigated by the real-time measurement of intracellular calcium signal changes, which was performed by the high-throughput CCD imaging plate reader FLIPR using Fluo-4D calcium ion fluorescent dye from Life Technologies. The specific test steps were as follows:

A. Ramos cells (ECACC) were seeded in a 384 experimental plate using cell culture medium (RPMI 1640 medium supplemented with 10% fetal bovine serum) at a density of 5×10⁶ cells/mL, 20 μL per well.

B. The compounds (compound (I) or CC-292) were diluted in DMSO in a 384 compound plate and transferred to the culture medium at 25 μL per well.

C. The cell plate and the compound plate were placed into FLIPR. 10 μL was transferred from the compound plate by the FLIPR instrument and added to the cell plate. The cell plate was removed and incubated in a 37° C. incubator for 1 h.

D. One hour later, 40 μL of Fluo-4D dye (Life Technologies, formulated as 0.5× with 40 mL of HBSS and HEPES buffer) was added to the cell plate and incubated at 37° C. for 50 min.

E. 10 μL of IgM at 8×EC80 concentration was added, and the plate was read using FLIPR.

F. The IC₅₀ results of the compounds were analyzed using EXCEL and Prism statistical software.

The results showed that compound (I) and CC-292 had a certain antagonistic effect, and compound (I) (IC₅₀ of 55±3.34 nM) was generally superior to CC-292 (IC₅₀ of 170.7±4.03 nM).

### 2. In Vivo Efficacy Study of Compound (I)

### 2.1 Evaluation of In Vivo Efficacy Model of Collagen-Induced Arthritis (CIA) in Mice

In this study, the vehicle or solvent was prepared at a volume ratio of DMSO:40% HP-β-CD:water being 5:15:80 (10 ml of DMSO was mixed well with 30 ml of 40% HP-β-CD and 160 ml of MilliQ pure water and then divided into portions).

In this study, compound (I) (or test compound (I)) was prepared by weighing a certain amount of the compound represented by Formula (I), adding a calculated volume of DMSO, and mixing with shaking until the mixture dissolved. Then, DMSO solution, 40% HP-β-CD, and MilliQ pure water were added (volume ratio of DMSO:40% HP-β-CD:water=5:15:80). The solution was thoroughly mixed and diluted three-fold to the administration concentration. Finally, compound (I) at a specific administration concentration was obtained.

In this study, CC-292 solution was prepared by weighing a certain amount of CC-292 compound (purity: 98.7%), adding the calculated volume of DMSO, and mixing with shaking until the mixture dissolved. Then, DMSO solution, 40% HP-β-CD, and MilliQ pure water were added (volume ratio of DMSO:40% HP-β-CD:water=5:15:80). The solution was thoroughly mixed and diluted three-fold to the administration concentration. Finally, CC-292 at a specific administration concentration was obtained.

The model of collagen-induced arthritis in mice was a classic animal model used to evaluate the efficacy of medicaments for rheumatoid arthritis (RA), and the pathogenesis and symptoms were significantly correlated with RA disease. Clinical symptoms of arthritis were induced in mice by injecting collagen emulsion into the tail of mice. The specific steps were as follows:

DBA/1 mice were randomly divided into 6 groups, with 10 mice in each group except the blank control group (5 mice). The mice in the normal group did not need to be immunized, while the mice in the other groups were all immunized. The day of the first immunization was recorded as day 0, and the subsequent days were marked in sequence. DBA/1 mice were anesthetized with isoflurane and injected subcutaneously with 50 μl of the prepared collagen emulsion (containing 200 μg of CII) at the tail (2 cm to 3 cm from the base of the tail). On day 21, the same volume of collagen emulsion was injected into the tail in the same way.

On day 28, when the average clinical score reached 1 to 2, the mice were randomly redivided into 4 treatment groups (10 mice per group) and a blank vehicle control group (10 mice per group) according to body weight and score. The relevant dosage design of the test compound (I) and the control compound CC-292 was determined based on the results of the preliminary experiments. The first group was a normal group (not fed with any medicament or vehicle); the second group was given a blank vehicle control (the vehicle was administered at 10 mL/kg); the third group was given CC-292 at a dose of 3 mg/kg (CC-292 concentration of 0.3 mg/mL, and an administration volume of 10 mL/kg); the fourth, fifth, and sixth groups were given compound (I) at doses of 1 mg/kg (compound (I) concentration of 0.1 mg/mL, and an administration volume of 10 mL/kg), 3 mg/kg (compound (I) concentration of 0.3 mg/mL, and an administration volume of 10 mL/kg), and 10 mg/kg (compound (I) concentration of 1 mg/mL, and an administration volume of 10 mL/kg), respectively. The administration was performed once daily for 14 days by gavage at 10 mL/kg. After the administration, the clinical score curve of each animal in each group was analyzed to calculate the area under the curve (AUC). The inhibition rate of each administration group relative to the blank vehicle control was calculated based on the average AUC between groups.

Clinical observation: From 7 days before immunization to 21 days after immunization, the basic health status and weight changes of DBA/1 mice were observed daily (recorded twice a week). After day 22, the health status, disease incidence, and weight changes of the mice were observed daily (recorded every two days) until the end of the experiment.

Clinical scoring: After boosting immunization, mice were observed for disease progression every day. When mice began to develop symptoms of arthritis (clinical symptoms of arthritis appeared), they were scored on a scale from 0 to 4 based on the severity of the lesions (redness, swelling, joint deformation), with a maximum score of 4 for each limb and a maximum score of 16 for each animal. The scoring was performed at least three times a week.

Pathology: On day 42, mice were euthanized. The two hind limbs of the mice were taken, soaked in 10% formalin solution, decalcified with formic acid solution, embedded in paraffin, sectioned, stained with hematoxylin-eosin (HE), and observed under a microscope. The degree of joint damage was evaluated from four aspects: inflammatory cell infiltration, pannus formation, cartilage damage, and bone resorption, and scored on a scale from 0 to 4.

The experimental results showed that compared with the normal group, the average body weight of each immune group decreased significantly after administration. After day 33, the average body weight of the 1 mg/kg compound (I) treatment group remained basically unchanged, while the average body weight of the 3 mg/kg and 10 mg/kg compound (I) treatment groups increased. In addition, the body weight of the 10 mg/kg group showed significant differences compared with the blank vehicle control group on days 37 and 40, indicating a good weight recovery effect.

The inflammation inhibition rate of the 1 mg/kg compound (I) administration group was 65.5%, the inhibition rate of the 3 mg/kg compound (I) administration group was 82.4%, and the inhibition rate of the 10 mg/kg compound (I) administration group was 83.9%. Compared with the blank vehicle control, the three administration doses showed extremely significant differences (p<0.001) and a certain dose-effect dependence. The inflammation inhibition rate of the 3 mg/kg CC-292 administration group was 29.1%. Compared with the blank vehicle control, no significant difference was observed in the 3 mg/kg CC-292 administration group.

The pathological study showed that only mild inflammation was observed in the foot joint bone tissue of the three dose groups (1, 3, and 10 mg/kg) of compound (I). The histopathological scores decreased with increasing doses, showing a dose-dependent manner. Compared with the blank vehicle control group, the histopathological scores were significantly different. The reference control 3 mg/kg CC-292 administration group showed certain inflammation and more bone tissue damage, and its histopathological score was not significantly different from that of the blank vehicle control group.

### 2.2 Evaluation of In Vivo Efficacy Model of PG-PS-Induced Arthritis in Rats

In this study, the vehicle or solvent was prepared at a volume ratio of DMSO:40% HP-β-CD:water of 5:15:80.

In this study, compound (I) (or test compound (I)) was prepared by weighing a certain amount of the compound represented by Formula (I), adding a calculated volume of DMSO, and mixing with shaking until the mixture dissolved. Then, DMSO solution, 40% HP-β-CD, and MilliQ pure water were added (volume ratio of DMSO:40% HP-β-CD:water=5:15:80). The solution was thoroughly mixed. Finally, compound (I) at a specific administration concentration was obtained.

In this study, CC-292 solution was prepared by weighing 15 mg of CC-292 compound (purity: 98.7%), dissolving the CC-292 compound in 2.5 mL of DMSO, and mixing with 7.5 mL of 40% HP-β-CD and 40 mL of deionized water. Finally, CC-292 at a concentration of 3 mg/10 mL was obtained. Intraperitoneal injection of PG-PS suspension can effectively induce chronic, erosive, and relapsing symptoms similar to human rheumatoid arthritis in rats, and is often used to evaluate the in vivo effectiveness of medicaments in the early discovery of arthritis medicaments. The specific steps were as follows:

On day 0, Lewis rats were randomly divided into 6 groups, with 5 rats in the first group and 10 rats in each group from the second group to the sixth group. All rats in the second to sixth groups were intraperitoneally injected with PG-PS (Lee Laboratories, PG-PS 10S, 5.1 mg/ml Rhamnose) to establish the model. The PG-PS injection dose v=15 μg/g×bw (rat weight, in g)/5.1 μg/μL.

On day 1, the model rats (the second to sixth groups) were randomly redivided into 5 groups, with 10 rats in each group. The animals in each group were given the corresponding medicament or vehicle by gavage once a day until the 29th day. The first group was the normal group, with a dose of 1 mL/kg (vehicle); the second group was given a blank vehicle control, with a dose of 1 mL/kg (vehicle); the third group was given CC-292, with a dose of 3 mg/kg (CC-292 concentration of 0.3 mg/mL, and an administration volume of 10 mL/kg); the fourth, fifth, and sixth groups were given compound (I), with doses of 1 mg/kg (compound (I) concentration of 0.1 mg/mL, and an administration volume of 10 mL/kg), 3 mg/kg (compound (I) concentration of 0.3 mg/mL, and an administration volume of 10 mL/kg), and 10 mg/kg (compound (I) concentration of 1 mg/mL, and an administration volume of 10 mL/kg), respectively. Clinical observations and clinical scoring were performed daily during the administration period. The ankle diameter of each animal after modeling was subtracted from its ankle diameter before modeling to obtain the ankle diameter difference (delta ankle diameter). The area under the curve (AUC) was calculated by analyzing the ankle diameter difference of each animal in each group. The inhibition rate of each administration group relative to the blank vehicle control was calculated by the average AUC between groups.

Pathology: On day 30, the rats were euthanized. The two hind limbs of rats were stained with toluidine blue to evaluate the degree of joint damage in terms of inflammatory cell lesions, pannus formation, cartilage damage, bone resorption, and periosteal new bone formation, and the severity was scored on a scale from 0 to 7.

The experimental results showed that the inhibition rate of the 1 mg/kg compound (I) administration group was 47.7%, the inhibition rate of the 3 mg/kg compound (I) administration group was 64.7%, and the inhibition rate of the 10 mg/kg compound (I) administration group was 77.3%. Compared with the blank vehicle control, the administration doses of 3 mg/kg and 10 mg/kg showed extremely significant differences (p<0.001) and a certain dose-effect dependence. The 1 mg/kg compound (I) administration group also showed a significant difference (p<0.01). The inhibition rate of the 3 mg/kg CC-292 administration group was −0.1%. Compared with the blank vehicle control, the 3 mg/kg CC-292 administration group showed no inhibitory trend.

The pathological study showed that the 3 mg/kg compound (I) administration group and the 10 mg/kg compound (I) administration group had a significant improvement effect on joint tissue lesions, with significant differences in the scoring results (p<0.05). The 1 mg/kg compound (I) administration group showed a trend of alleviating tissue damage, but no significant difference was observed. The tissue lesions in the 3 mg/kg CC-292 administration group were more severe, and the average score was slightly lower than that in the blank vehicle control group, but there was no significant difference.

### 2.3 Efficacy Evaluation in the Passive Cutaneous Anaphylaxis (PCA) Animal Model

Passive cutaneous anaphylaxis test, also known as PCA reaction, is a local allergic reaction caused by antibodies that bind to the same or different animal tissues. The PCA animal model in the present disclosure was a passive sensitization model in which IgE binds to specific receptors on skin mast cells. When allergens were stimulated, local mast cells released allergic mediators, thereby increasing local vascular permeability. It was often used as an efficacy model for urticaria indications and can also demonstrate the anti-allergic effect of medicaments in the pathogenesis of skin inflammation and eczema. The specific steps were as follows:

In this step, compound (I) was prepared as follows. The solvent was 5% DMSO (dimethyl sulfoxide)+15% 40% HP-β-CD (hydroxypropyl-β-cyclodextrin)+10% 0.4% Kolliphor EL (commercially available)+70% water. A certain amount of the compound represented by Formula (I) was weighed, dissolved in the calculated volume of DMSO, and then thoroughly mixed with the calculated volume of HP-β-CD, Kolliphor EL with deionized water. Finally, compound (I) at the administration concentration was obtained.

In this step, prednisone was prepared with 0.5% CMC-Na (sodium carboxymethylcellulose).

In this step, cetirizine was prepared as follows. The solvent was 5% DMSO (dimethyl sulfoxide)+15% 40% HP-β-CD (hydroxypropyl-β-cyclodextrin)+10% 0.4% Kolliphor EL (commercially available)+70% water. A certain amount of cetirizine was weighed, dissolved in the calculated volume of DMSO, and then thoroughly mixed with the calculated volume of HP-β-CD, Kolliphor EL with deionized water. Finally, cetirizine at the administration concentration was obtained (cetirizine in the subsequent examples were all cetirizine containing the solvent).

In this step, LOU-064 was prepared as follows. The solvent was 5% DMSO (dimethyl sulfoxide)+15% 40% HP-β-CD (hydroxypropyl-β-cyclodextrin)+10% 0.4% Kolliphor EL (commercially available)+70% water. A certain amount of LOU-064 was weighed, dissolved in the calculated volume of DMSO, and then thoroughly mixed with the calculated volume of HP-β-CD, Kolliphor EL and deionized water. Finally, the LOU-064 solution at the administration concentration was obtained. LOU-064 was adjusted to a pH that obtained a clarified solution.

The PCA model in mice was induced using anti-DNP IgE. Specifically, BALB/c mice were divided into 9 groups, namely, a normal control group (no administration, injection, or feeding of any solvent), a model group (no administration, injection, or feeding of any solvent), a prednisone group (10 mg/kg, administered for 3 consecutive days, QD (once a day), prednisone concentration of 1 mg/mL, and an administration volume of 10 mL/kg), a cetirizine group (20 mg/kg, administered for 3 consecutive days, QD, cetirizine concentration of 2 mg/mL, and an administration volume of 10 mL/kg), a low-dose group and a medium-dose group for the test substance LOU-064 (15 mg/kg and 45 mg/kg, administered for 3 consecutive days, BID (twice a day), LOU-064 concentrations of 1.5 mg/mL and 4.5 mg/mL, and an administration volume of 10 mL/kg), and a low-dose group, a medium-dose group, and a high-dose group for compound (I) (15 mg/kg, 45 mg/kg, and 90 mg/kg, administered for 3 consecutive days, BID, compound (I) concentrations of 1.5 mg/mL, 4.5 mg/mL, and 9 mg/mL, and an administration volume of 10 mL/kg), with 9 mice in each group, administered by gavage for a total of 4 days. On day 3 of the administration, anti-DNP IgE (Sigma-Aldrich) was injected into the left ears of 8 groups of mice (no normal control group), and the same volume of PBS was injected into the right ears of 8 groups and the left and right ears of the normal control group to allow it to bind to mast cells. Then, 24 hours after the fourth day of anti-DNP IgE injection, a mixed solution of antigen (DNP-HAS, Sigma) and pigment Evan's Blue (Sigma) (0.25 mL/mouse, i.v.) was injected into the tail vein (the normal control group was injected with the same volume of the mixed solution of antigen and pigment Evan's Blue). The antigen reacted with the antibody attached to the mast cells, causing a local allergic reaction, increased local vascular permeability, and pigment penetration into the tissue. The reaction intensity was determined by measuring the local pigment content 30 minutes later. The dye was extracted from the left and right ears, and the absorbance of Evan's blue was measured at a specific wavelength. The content of Evan's blue was calculated according to the standard curve (see Table 1, where the concentration of Evan's Blue in the right ear of each group was not significantly different from that in the left ear of the normal control group). The efficacy effects of each test substances in the PCA animal model were evaluated.

TABLE 1

| | Concentration |
|---|---|
| | of Evan's Blue |
| | (μg/mL) |
| Groups | Left ear |

| | | |
|---|---|---|
| Normal control group | Average value | 16.61 |
| | Standard error | 1.34 |
| Model group | Average value | 43.35 |
| | Standard error | 3.68 |
| Prednisone group (10 mg/kg) | Average value | 25.36 |
| | Standard error | 2.13 |
| Cetirizine group (20 mg/kg) | Average value | 22.70 |
| | Standard error | 2.82 |
| LOU-064 (15 mg/kg) | Average value | 24.45 |
| | Standard error | 1.76 |
| LOU-064 (45 mg/kg) | Average value | 17.24 |
| | Standard error | 1.53 |
| Compound (I) (15 mg/kg) | Average value | 25.68 |
| | Standard error | 1.97 |
| Compound (I) (45 mg/kg) | Average value | 18.21 |
| | Standard error | 1.19 |
| Compound (I) (90 mg/kg) | Average value | 22.00 |
| | Standard error | 2.18 |

The experimental results showed that compared with the normal control group, the weight of the ear on the modeling side (left side) of the mice in the model group was significantly increased, and the Evan's blue content in the ear was significantly increased. Compared with the model group, the Evan's Blue content in the modeling ear of the model animals in three dose groups of compound (I) was significantly reduced, with significant efficacy. The low-dose group and the medium-dose group showed a dose-effect relationship, and the effect was comparable to that of LOU-064 at the same dose. The low-dose group was comparable to the prednisone and cetirizine groups. Therefore, compound (I) has a good therapeutic effect on anti-DNP IgE-induced passive cutaneous anaphylaxis in mice.

### Example 2: Clinical Trial Study of Compound (I) Capsule

### 1. Trial Method

In this example, 96 healthy Chinese aged 18 to 45 were recruited as subjects. The trial included 8 dose groups, each with 12 subjects, who were randomly divided into groups, 10 of whom received compound (I) capsules (i.e., the trial group) and 2 received placebo capsules (containing no compound (I), i.e., the control group or placebo group). Compound (I) capsules in this example were prepared by filling the capsules with ingredients containing compound (I); and the difference between the placebo capsules and compound (I) capsules was only that the placebo capsules did not contain compound (I) ingredient. The dosages involved in this example were all the amount of compound (I). Based on safety and exploration factors, the human MRSD (maximum recommended starting dose) of compound (I) capsule used in the example was set at 10 mg, and the human maximum recommended dose was set at 800 mg. A total of 8 dose groups were set up between the initial dose and the maximum dose (see Table 2 for the dose escalation scheme of the clinical trial): 10 mg, 25 mg, 50 mg, 100 mg, 200 mg, 400 mg, 600 mg, and 800 mg, and single administration safety and tolerability trials were conducted group by group.

The subjects fasted for 10 h before administration and took a single oral administration of compound (I) capsule or placebo capsule at the prescribed dose on the morning of the trial day. Water was prohibited within 2 h after the administration and food was prohibited within 4 h after the administration. Blood sample collection: 3.0 ml of venous blood was collected before administration (within 45 min) and at 10 min, 20 min, 0.5 h, 1 h, 1.5 h, 1.75 h, 2 h, 2.5 h, 3 h, 3.5 h, 4 h, 6 h, 8 h, 12 h, 16 h, 24 h, 36 h, 48 h, and 72 h after administration. Urine sample collection: Urine samples were collected before administration (within 2 h) and at 0-2 h, 2-4 h, 4-8 h, 8-12 h, 12-16 h, 16-24 h, 24-48 h, and 48-72 h after administration. Feces collection: Feces were collected before administration (within 3 days) and at 0-24 h, 24-48 h, and 48-72 h after administration.

TABLE 2
Dose escalation scheme for Phase I single-dosing
clinical trial of compound (I) capsules

| | | Dose | Number of | Number of |
|---|---|---|---|---|
| | Administration | escalation | people in | people in |
| Groups | dose (mg) | ratio | trial group | control group |
| 1 | 10 | — | 10 | 2 |
| 2 | 25 | 150% | 10 | 2 |
| 3 | 50 | 100% | 10 | 2 |
| 4 | 100 | 100% | 10 | 2 |
| 5 | 200 | 100% | 10 | 2 |
| 6 | 400 | 100% | 10 | 2 |
| 7 | 600 | 50% | 10 | 2 |
| 8 | 800 | 33% | 10 | 2 |

### 2. Statistical Analysis Methods

### 2.1 Statistical Analysis of Pharmacokinetics

Per-protocol set: All subjects who met the inclusion criteria, did not meet the exclusion criteria, had no major protocol deviations, and completed administration, pharmacokinetic and pharmacodynamic sampling, and safety assessments according to the protocol constituted the Per-protocol set of this study.

The analysis was performed using the Per-protocol set. The time-dependent plasma concentration data of compound (I) capsules were processed using WinNonlin version 6.4. The main pharmacokinetic parameters of each subject were calculated using a non-compartmental model, and the linear relationship between dose and Cₘₐₓ and AUC₀₋ₜ was analyzed using a power function model.

### 2.2 Statistical Analysis of Pharmacodynamics

The receptor occupancy of compound (I) in human peripheral blood mononuclear cell lysate was detected by ELISA. The analysis method was to perform relative quantitative determination of BTK in human peripheral blood mononuclear cells (PBMCs) using ELISA.

PBMC lysate containing BTK was incubated with compound (I), with a control containing no compound (I), then incubated with biotinlated compound (I), and then added to a well plate pre-coated with streptavidin. When compound (I) was incubated with PBMC lysate, it was bound to BTK, thereby preventing BTK from binding to biotinylated compound (I). Since the complex cannot bind to the well plate, it was difficult to generate a response signal.

Rabbit anti-BTK primary antibody incubated with the above samples would bind to BTK. Tetramethylbenzidine (TMB) was the substrate of HRP coupled to the anti-rabbit secondary antibody. The reaction was terminated by sulfuric acid. After color development, the absorbance was measured at a wavelength of 450 nm. 620 nm can be used for background correction. The content of free BTK was proportional to the absorbance. In this way, the content of BTK can be indirectly detected. The data was processed as follows:

$$Free BTK ( \% ) = 100 \% \times [ ( {S}_{sample X} - {S}_{sample X + compound ( I )} ) / ( {S}_{before administration on day 1} - {S}_{before administration on day 1 + compound ( I )} ) ] .$$

Note: S_{sampleX}: represents the absorbance value of the sample X to be tested;
S_{sample X+compound (I)}: represents the absorbance value of the sample X to be tested after adding compound (I) [10 uM], which serves as the background reference of the sample;
S_{before administration on day 1}: represents the signal value of the sample before the first administration; and
S_{before administration on day 1+compound (I)}: represents the absorbance value of the sample before the first administration after adding compound (I) [10 uM], which serves as the background reference of the sample.

$$BTK occupancy rate \% = 100 \% - free BTK \% .$$

The analysis was performed using the Per-protocol set. The differences in BTK target occupancy rate among the different dose groups was analyzed using the Chi-Square test; and the relationship between dose, plasma concentration, and BTK target occupancy rate was analyzed using Logistic Regression.

### 2.3 Assessment and Statistical Analysis of Safety and Tolerability

Safety data set: including all subjects who received at least one administration and had post-administration safety data. Safety evaluation indicators include adverse events/adverse reactions, laboratory tests, electrocardiograms, vital signs, physical examination, thyroid color Doppler ultrasound, upper abdominal CT, and chest X-ray. All subjects in the placebo group were combined into one group. A safety analysis set was used, and statistical descriptions were mainly used for safety indicators. Comparative analyses were conducted before and after treatment in each treatment group, between different dose trial groups, and between the trial group and the placebo group.

### 3. Analysis of Results

### 3.1 Pharmacokinetics

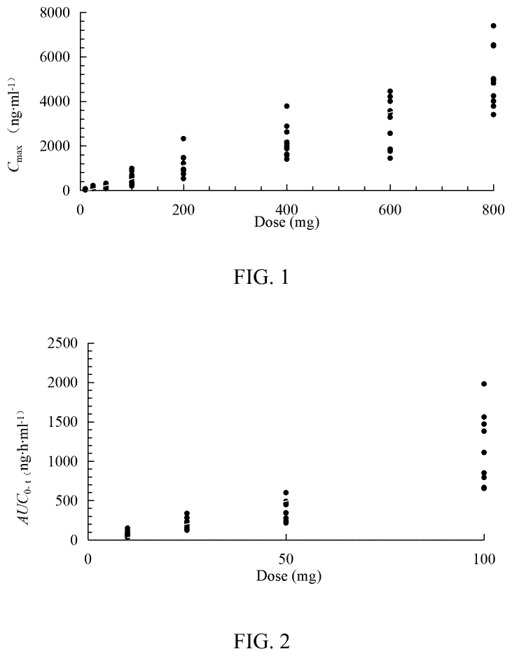

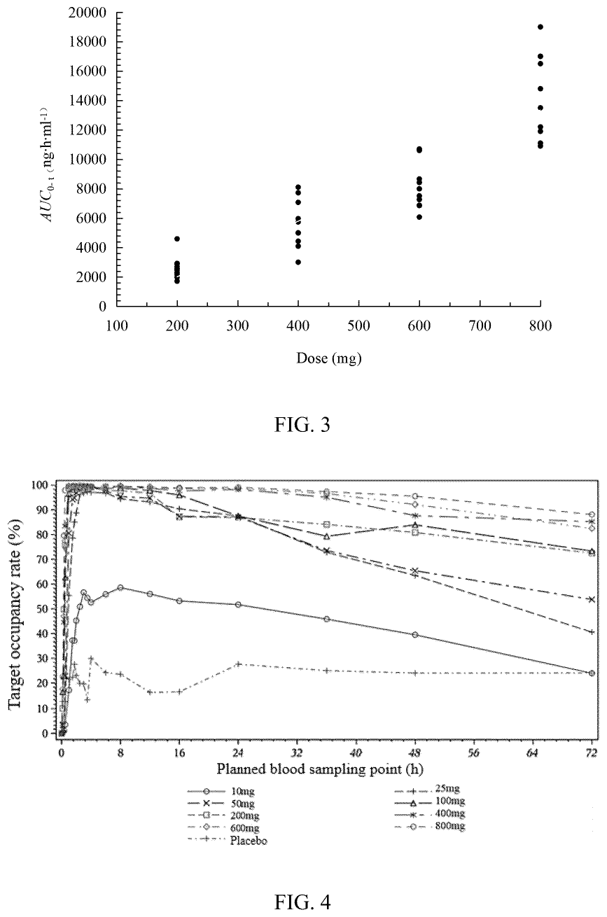

After single-dosing administration of compound (I) capsules to healthy subjects, there were statistically significant differences in CL/F, Vz/F, T_{1/2}, MRT₀₋ₜ, MRT_{0-∞}, and Tₘₐₓ of compound (I) capsules among the eight dose groups (p<0.05), but no statistically significant differences in CLr (p>0.05) (see Table 3 for details). In the dosage range from 10 mg to 800 mg, βCₘₐₓ of compound (I) capsules was 1.0740, which was in the range from 0.8 to 1.25. The 90% confidence interval of the linear equation slope βCₘₐₓ ranged from 1.0247 to 1.1234, partially falling within the judgment interval from 0.9491 to 1.0509, which indicates that within the dose range from 10 mg to 800 mg, the Cₘₐₓ of compound (I) capsules in Chinese healthy subjects had a linear pharmacokinetic trend (see FIG. 1 and Table 4 for details). Furthermore, AUC₀₋ₜ showed a linear pharmacokinetic trend in the ranges from 10 mg to 100 mg (FIG. 2 and Table 5) and from 200 mg to 800 mg (FIG. 3 and Table 6).

TABLE 3
Pharmacokinetic parameters of compound (I) capsules after administration

| PK | 10 mg | 25 mg | 50 mg | 100 mg | 200 mg | 400 mg | 600 mg | 800 mg |
|---|---|---|---|---|---|---|---|---|
| parameters | (n = 10) | (n = 10) | (n = 10) | (n = 10) | (n = 10) | (n = 10) | (n = 10) | (n = 10) |
| Tₘₐₓ(h) | 1.03 ± | 1.03 ± | 1.25 ± | 1.40 ± | 1.45 ± | 1.80 ± | 2.00 ± | 1.55 ± |
| | 0.650 | 0.381 | 0.755 | 0.738 | 0.599 | 0.387 | 1.00 | 0.956 |
| Cₘₐₓ | 46.1 ± | 115 ± | 189 ± | 538 ± | 1140 ± | 2200 ± | 3060 ± | 5060 ± |
| (ng/ml) | 15.3 | 39.0 | 79.5 | 258 | 510 | 722 | 1090 | 1330 |
| AUC₀₋ₜ | 77.7 ± | 207 ± | 387 ± | 1112 ± | 2615 ± | 5609 ± | 8088 ± | 14392 ± |
| (h*ng/ml) | 33.0 | 65.6 | 124 | 464 | 794 | 1640 | 1544 | 2877 |
| AUC_{0-∞} | 80.8 ± | 209 ± | 391 ± | 1116 ± | 2623 ± | 5628 ± | 8118 ± | 14492 ± |
| (h*ng/ml) | 33.8 | 65.8 | 123 | 464 | 793 | 1635 | 1530 | 2803 |
| AUC_{_{—}}_{% Extrap} | 3.9 ± | 1.3 ± | 1.1 ± | 0.4 ± | 0.3 ± | 0.4 ± | 0.4 ± | 0.8 ± |
| (%) | 1.8 | 0.7 | 1.1 | 0.2 | 0.4 | 0.4 | 0.5 | 2.2 |
| λ_{z} (1/h) | 0.866 ± | 0.744 ± | 0.743 ± | 0.413 ± | 0.323 ± | 0.148 ± | 0.0995 ± | 0.178 ± |
| | 0.201 | 0.209 | 0.171 | 0.193 | 0.143 | 0.07 | 0.0504 | 0.104 |
| T_{1/2} (h) | 0.847 ± | 1.00 ± | 0.997 ± | 2.00 ± | 2.71 ± | 5.45 ± | 8.67 ± | 4.79 ± |
| | 0.225 | 0.299 | 0.331 | 0.859 | 1.61 | 2.01 | 4.47 | 2.08 |
| V_{z}/F (ml) | 169761 ± | 180724 ± | 197180 ± | 301076 ± | 31170 ± | 63611 ± | 99639 ± | 385448 ± |
| | 71217 | 50498 | 77486 | 181046 | 179934 | 387352 | 675744 | 154119 |
| CL/F (ml/h) | 144573 ± | 130484 ± | 141463 ± | 103963 ± | 81348 ± | 77293 ± | 76135 ± | 57109 ± |
| | 63769 | 41046 | 49204 | 39830 | 20028 | 25109 | 13299 | 11074 |
| MRTₗₐₛₜ (h) | 1.53 ± | 1.80 ± | 1.99 ± | 2.30 ± | 2.59 ± | 3.13 ± | 4.01 ± | 3.97 ± |
| | 0.331 | 0.347 | 0.548 | 0.585 | 0.332 | 0.689 | 0.953 | 0.990 |
| MRT_{INF}_{_{—}}_{obs} | 1.69 ± | 1.87 ± | 2.07 ± | 2.36 ± | 2.67 ± | 3.25 ± | 4.25 ± | 4.04 ± |
| (h) | 0.356 | 0.351 | 0.599 | 0.579 | 0.355 | 0.725 | 1.11 | 0.909 |
| CLr (ml/h) | 934 ± | 1196 ± | 789 ± | 767 ± | 792 ± | 946 ± | 868 ± | 932 ± |
| | 213 | 476 | 263 | 179 | 225 | 391 | 267 | 331 |

Note:
Data are presented as mean ± standard deviation.

Cₘₐₓ represents peak concentration; Tₘₐₓ represents time to peak concentration; λ_{z} represents terminal elimination rate constant; and AUC represents area under the plasma concentration-time curve, where AUC₀₋ₜ represents area under the plasma concentration-time curve from time zero to the last quantifiable blood sampling point, AUC_{0-∞} represents area under the plasma concentration-time curve from time zero to infinity, and AUC_{_%Extrap} represents residual area percentage (AUC_{_%Extrap}=[(AUC_{0-∞}−AUC₀₋ₜ)/AUC_{0-∞}]×100%). T_{1/2} represents terminal elimination half-life, V_{z}/F represents apparent volume of distribution in the terminal phase after non-intravenous administration, CL/F represents apparent systemic clearance of the medicament from plasma after oral administration, and MRTₗₐₛₜ represents mean residence time from zero to the minimum detectable plasma concentration. MRT_{INF_obs} represents mean residence time extrapolated from zero to infinity, and CLr represents renal clearance of the medicament from plasma.

TABLE 4
Linear evaluation results of power function model of
Cₘₐₓ and dose at doses ranging from 10 mg to 800 mg
for compound (I) capsules after single administration

| | Estimated | | | |
|---|---|---|---|---|
| Parameter | value | SE | 90% CI | t-value |
| β_{Cmax} | 1.0740 | 0.0296 | 1.0247-1.1234 | 36.2457 |

| | |
|---|---|
| Judgment interval | 0.9491-1.0509 |
| Judgment criteria | 0.8-1.25 |

Power function model formula: PK=α*dose^{β}.

Judgment interval calculation formula: 1+Ln(θ_{L}/Ln(r)<β<1+ln(θ_{H})/ln(r); where r=D_{H}/D_{L}, D_{H} represents high dose, D_{L} represents low dose, θ_{L}=0.8, and θ_{H}=1.25.

TABLE 5
Linear evaluation results of power function model of AUC₀₋ₜ
and dose at doses ranging from 10 mg to 100 mg of compound
(I) capsules after single administration to healthy subjects

| | Estimated | | | |
|---|---|---|---|---|
| Parameter | value | SE | 90% CI | t-value |
| β_{AUC} | 1.1267 | 0.0696 | 1.0094-1.2441 | 16.1845 |

| | |
|---|---|
| Judgment interval | 0.9031-1.0969 |
| Judgment criteria | 0.8-1.25 |

Power function model formula: PK=α*dose^{β}.

Judgment interval calculation formula: 1+Ln(θ_{L}/Ln(r)<β<1+ln(θ_{H})/ln(r); where r=D_{H}/D_{L}, D_{H} represents high dose, D_{L} represents low dose, θ_{L}=0.8, and θ_{H}=1.25.

TABLE 6
Linear evaluation results of power function model of AUC₀₋ₜ
and dose at doses ranging from 200 mg to 800 mg of compound
(I) capsule after single administration to healthy subjects

| | Estimated | | | |
|---|---|---|---|---|
| Parameter | value | SE | 90% CI | t-value |
| β_{AUC} | 1.1940 | 0.0776 | 1.0632-1.3249 | 15.3802 |

| | |
|---|---|
| Judgment interval | 0.8390-1.1610 |
| Judgment criteria | 0.8-1.25 |

Power function model formula: PK=α*dose^{β}.

Judgment interval calculation formula: 1+Ln(θ_{L}/Ln(r)<β<1+ln(θ_{H})/ln(r); where r=D_{H}/D_{L}, D_{H} represents high dose, D_{L} represents low dose, θ_{L}=0.8, and θ_{H}=1.25.

### 3.2 Pharmacodynamics

The maximum BTK target occupancy rate was 58.93% after a single administration of 10 mg of compound (I) capsules, and close to 100% at a single administration of 25 mg or above (see FIG. 4 for details). The time to peak ranged from 0.5 h to 4 h, and it gradually shortened with increasing dose. After administration of the placebo group, the BTK target occupancy rate was maintained between 20% and 30% for most of the time.

### 3.3 Relationship Between Pharmacokinetics and Pharmacodynamics

The quantitative relationship between pharmacokinetics and pharmacodynamics was analyzed based on the pharmacokinetic and pharmacodynamic data obtained for each dose.

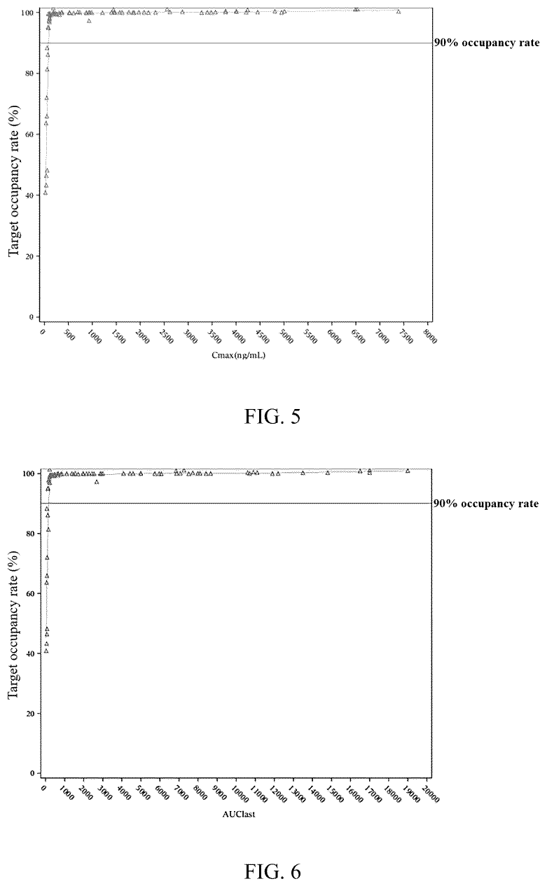

Data fitting shows that under lower Cₘₐₓ and AUCₗₐₛₜ conditions, the BTK target occupancy rate can reach nearly 100% (see FIG. 5 and FIG. 6 for details).

### 3.4 Safety

A total of 44 adverse events (AE) occurred in 29 of the 96 subjects, of which 1 was a pre-administration adverse event and the remaining 43 were treatment-emergent adverse events (TEAE). A total of 35 TEAEs occurred in 25 subjects (31.3%) in compound (I) group, and a total of 8 TEAEs occurred in 4 subjects (25%) in the placebo group. Except for one subject in the placebo group who experienced a grade 2 AE of anemia, the rest were grade 1 AEs (see Table 7 for details).

In the trial group, except for 2 AEs (upper respiratory tract infection, eyelid surgery) that were treated with medication and 1 AE (upper respiratory tract infection) that was treated with a medical antipyretic patch, no measures were taken for the remaining AEs and they recovered to normal or baseline levels on their own. Except for 2 cases of nephrolithiasis AE which were definitely not related to the trial medicament and remained unchanged, all other AE outcomes disappeared. 10 AEs occurring in 9 subjects were judged to be possibly related to the trial medicament and were adverse reactions. The incidence of adverse events in the 50, 100, 200, and 400 mg dose groups was higher, all at 50%. No adverse events occurred in the 800 mg dose group. The incidence of AEs in other dose groups ranged from 10% to 30%. The incidence of adverse events had no obvious dose-related relationship. In the placebo group, except for one AE (anemia) that was treated with medication and one AE (dizziness) in which the subject took oral glucose, no measures were taken for the remaining AEs and they recovered to normal on their own. All AE outcomes were disappeared. The incidence of adverse reactions was 12.5% (2/16). No SAEs (serious adverse events) occurred in this trial, and no AEs leading to withdrawal from the trial occurred (see Tables 8 and 9 for details).

TABLE 7
Summary of incidence of adverse events during the trial

| | 10 | 25 | 50 | 100 | 200 | 400 | 600 | 800 | Trial | Placebo |
|---|---|---|---|---|---|---|---|---|---|---|
| | mg | mg | mg | mg | mg | mg | mg | mg | group | group |
| | (n = 10) | (n = 10) | (n = 10) | (n = 10) | (n = 10) | (n = 10) | (n = 10) | (n = 10) | (n = 80) | (n = 16) |
| | n (%) | n (%) | n (%) | n (%) | n (%) | n (%) | n (%) | n (%) | n (%) | n (%) |
| Adverse events | 1 | 1 | 5 | 5 | 5 | 5 | 3 | 0 | 25 | 4 |
| | (10.0) | (10.0) | (50.0) | (50.0) | (50.0) | (50.0) | (30.0) | (0) | (31.3) | (25.0) |
| Adverse reactions | 1 | 1 | 1 | 1 | 1 | 4 | 0 | 0 | 9 | 2 |
| | (10.0) | (10.0) | (10.0) | (10.0) | (10.0) | (40.0) | (0) | (0) | (11.3) | (12.5) |
| Grade 1^{a} | 1 | 1 | 5 | 5 | 5 | 5 | 3 | 0 | 25 | 3 |
| | (10.0) | (10.0) | (50.0) | (50.0) | (50.0) | (50.0) | (30.0) | (0) | (31.3) | (18.8) |
| Grade 2^{a} | 0 | 0 | 0 | 0 | 0 | 0 | 0 | 0 | 0 | 1 |
| | (0) | (0) | (0) | (0) | (0) | (0) | (0) | (0) | (0) | (6.3) |
| Grade 3^{a} | 0 | 0 | 0 | 0 | 0 | 0 | 0 | 0 | 0 | 0 |
| | (0) | (0) | (0) | (0) | (0) | (0) | (0) | (0) | (0) | (0) |
| Serious adverse events | 0 | 0 | 0 | 0 | 0 | 0 | 0 | 0 | 0 | 0 |
| | (0) | (0) | (0) | (0) | (0) | (0) | (0) | (0) | (0) | (0) |
| Adverse events leading to | 0 | 0 | 0 | 0 | 0 | 0 | 0 | 0 | 0 | 0 |
| withdrawal from the trial | (0) | (0) | (0) | (0) | (0) | (0) | (0) | (0) | (0) | (0) |
| Adverse reactions leading | 0 | 0 | 0 | 0 | 0 | 0 | 0 | 0 | 0 | 0 |
| to withdrawal from the | (0) | (0) | (0) | (0) | (0) | (0) | (0) | (0) | (0) | (0) |
| trial | | | | | | | | | | |

^{a}The severity of adverse reactions was graded according to CTACE version 4.03.

TABLE 8
Incidence of systemic adverse events in subjects after administration

| | | | | | | | | | Trial | Placebo |
|---|---|---|---|---|---|---|---|---|---|---|
| | 10 mg | 25 mg | 50 mg | 100 mg | 200 mg | 400 mg | 600 mg | 800 mg | group | group |
| | (n = 10) | (n = 10) | (n = 10) | (n = 10) | (n = 10) | (n = 10) | (n = 10) | (n = 10) | (n = 80) | (n = 16) |
| | n (%) | n (%) | n (%) | n (%) | n (%) | n (%) | n (%) | n (%) | n (%) | n (%) |
| Total* | 1 (10.0) | 1 (10.0) | 5 (50.0) | 5 (50.0) | 5 (50.0) | 5 (50.0) | 3 (30.0) | 0 | 25 (31.3) | 4 (25.0) |
| Metabolic and | 0 | 0 | 0 | 0 | 0 | 0 | 0 | 0 | 0 | 1 (6.3) |
| nutritional | | | | | | | | | | |
| diseases | | | | | | | | | | |
| Decreased | 0 | 0 | 0 | 0 | 0 | 0 | 0 | 0 | 0 | 1 (6.3) |
| appetite | | | | | | | | | | |
| Infections and | 0 | 1 (10.0) | 0 | 0 | 1 (10.0) | 2 (20.0) | 0 | 0 | 4 (5.0) | 0 |
| infestations | | | | | | | | | | |
| Upper | 0 | 1 (10.0) | 0 | 0 | 1 (10.0) | 2 (20.0) | 0 | 0 | 4 (5.0) | 0 |
| respiratory tract | | | | | | | | | | |
| infection | | | | | | | | | | |
| Various | 0 | 0 | 0 | 0 | 0 | 1 (10.0) | 1 (10.0) | 0 | 2 (2.5) | 0 |
| inspections | | | | | | | | | | |
| Elevated white | 0 | 0 | 0 | 0 | 0 | 1 (10.0) | 0 | 0 | 1 (1.3) | 0 |
| blood cell count | | | | | | | | | | |
| Elevated | 0 | 0 | 0 | 0 | 0 | 1 (10.0) | 0 | 0 | 1 (1.3) | 0 |
| neutrophil | | | | | | | | | | |
| count | | | | | | | | | | |
| Abnormal | 0 | 0 | 0 | 0 | 0 | 0 | 1 (10.0) | 0 | 1 (1.3) | 0 |
| dorsalis pedis | | | | | | | | | | |
| artery pulse | | | | | | | | | | |
| Various | 0 | 0 | 2 (20.0) | 0 | 0 | 0 | 0 | 0 | 2 (2.5) | 1 (6.3) |
| neurological | | | | | | | | | | |
| diseases | | | | | | | | | | |
| Dizziness | 0 | 0 | 2 (20.0) | 0 | 0 | 0 | 0 | 0 | 2 (2.5) | 1 (6.3) |
| Various | 0 | 0 | 0 | 0 | 0 | 0 | 1 (10.0) | 0 | 1 (1.3) | 0 |
| musculoskeletal | | | | | | | | | | |
| and connective | | | | | | | | | | |
| tissue diseases | | | | | | | | | | |
| Musculoskeletal | 0 | 0 | 0 | 0 | 0 | 0 | 1 (10.0) | 0 | 1 (1.3) | 0 |
| pain | | | | | | | | | | |
| Various | 0 | 0 | 0 | 0 | 1 (10.0) | 0 | 0 | 0 | 1 (1.3) | 0 |
| surgeries and | | | | | | | | | | |
| medical | | | | | | | | | | |
| procedures | | | | | | | | | | |
| Eyelid surgery | 0 | 0 | 0 | 0 | 1 (10.0) | 0 | 0 | 0 | 1 (1.3) | 0 |
| Respiratory, | 1 (10.0) | 0 | 0 | 0 | 1 (10.0) | 0 | 0 | 0 | 2 (2.5) | 0 |
| thoracic, and | | | | | | | | | | |
| mediastinal | | | | | | | | | | |
| diseases | | | | | | | | | | |
| Nasal | 0 | 0 | 0 | 0 | 1 (10.0) | 0 | 0 | 0 | 1 (1.3) | 0 |
| congestion | | | | | | | | | | |
| Epistaxis | 1 (10.0) | 0 | 0 | 0 | 0 | 0 | 0 | 0 | 1 (1.3) | 0 |
| Psychiatric | 0 | 0 | 1 (10.0) | 0 | 0 | 0 | 1 (10.0) | 0 | 2 (2.5) | 0 |
| disorders | | | | | | | | | | |
| Hemophobia | 0 | 0 | 1 (10.0) | 0 | 0 | 0 | 0 | 0 | 1 (1.3) | 0 |
| Injection-site | 0 | 0 | 0 | 0 | 0 | 0 | 1 (10.0) | 0 | 1 (1.3) | 0 |
| phobia | | | | | | | | | | |
| Systemic | 0 | 0 | 0 | 0 | 0 | 0 | 0 | 0 | 0 | 1 (6.3) |
| diseases and | | | | | | | | | | |
| administration | | | | | | | | | | |
| site reactions | | | | | | | | | | |
| Fatigue | 0 | 0 | 0 | 0 | 0 | 0 | 0 | 0 | 0 | 1 (6.3) |
| Kidney and | 0 | 0 | 0 | 2 (20.0) | 0 | 0 | 0 | 0 | 2 (2.5) | 0 |
| urinary system | | | | | | | | | | |
| diseases | | | | | | | | | | |
| Nephrolithiasis | 0 | 0 | 0 | 2 (20.0) | 0 | 0 | 0 | 0 | 2 (2.5) | 0 |
| Gastrointestinal | 1 (10.0) | 0 | 1 (10.0) | 2 (20.0) | 1 (10.0) | 0 | 2 (20.0) | 0 | 7 (8.8) | 0 |
| diseases | | | | | | | | | | |
| Constipation | 0 | 0 | 1 (10.0) | 0 | 1 (10.0) | 0 | 0 | 0 | 2 (2.5) | 0 |
| Stomachache | 0 | 0 | 0 | 2 (20.0) | 0 | 0 | 1 (10.0) | 0 | 3 (3.8) | 0 |
| Diarrhea | 0 | 0 | 0 | 0 | 0 | 0 | 1 (10.0) | 0 | 1 (1.3) | 0 |
| Indigestion | 1 (10.0) | 0 | 0 | 0 | 0 | 0 | 0 | 0 | 1 (1.3) | 0 |
| Heart disease | 0 | 0 | 0 | 1 (10.0) | 0 | 2 (20.0) | 0 | 0 | 3 (3.8) | 1 (6.3) |
| Sinus | 0 | 0 | 0 | 1 (10.0) | 0 | 2 (20.0) | 0 | 0 | 3 (3.8) | 1 (6.3) |
| bradycardia | | | | | | | | | | |
| Blood and | 0 | 0 | 1 (10.0) | 0 | 2 (20.0) | 1 (10.0) | 1 (10.0) | 0 | 5 (6.3) | 3 (18.8) |
| lymphatic | | | | | | | | | | |
| system diseases | | | | | | | | | | |
| Anemia | 0 | 0 | 1 (10.0) | 0 | 2 (20.0) | 1 (10.0) | 1 (10.0) | 0 | 5 (6.3) | 3 (18.8) |
| Eye diseases | 0 | 0 | 0 | 0 | 0 | 1 (10.0) | 0 | 0 | 1 (1.3) | 0 |
| Amaurosis | 0 | 0 | 0 | 0 | 0 | 1 (10.0) | 0 | 0 | 1 (1.3) | 0 |

Note:
Adverse events were coded according to MedDRA V23.0.

TABLE 9
Adverse reactions to treatment

| | | | | | | | | | Trial | Placebo |
|---|---|---|---|---|---|---|---|---|---|---|
| | 10 mg | 25 mg | 50 mg | 100 mg | 200 mg | 400 mg | 600 mg | 800 mg | group | group |
| | (n = 10) | (n = 10) | (n = 10) | (n = 10) | (n = 10) | (n = 10) | (n = 10) | (n = 10) | (n = 80) | (n = 16) |
| | n (%) | n (%) | n (%) | n (%) | n (%) | n (%) | n (%) | n (%) | n (%) | n (%) |
| Total | 1 (10.0) | 1 (10.0) | 1 (10.0) | 1 (10.0) | 1 (10.0) | 4 (40.0) | 0 | 0 | 9 (11.3) | 2 (12.5) |
| Metabolic and | 0 | 0 | 0 | 0 | 0 | 0 | 0 | 0 | 0 | 1 (6.3) |
| nutritional | | | | | | | | | | |
| diseases | | | | | | | | | | |
| Decreased | 0 | 0 | 0 | 0 | 0 | 0 | 0 | 0 | 0 | 1 (6.3) |
| appetite | | | | | | | | | | |
| Infections and | 0 | 1 (10.0) | 0 | 0 | 1 (10.0) | 2 (20.0) | 0 | 0 | 4 (5.0) | 0 |
| infestations | | | | | | | | | | |
| Upper | 0 | 1 (10.0) | 0 | 0 | 1 (10.0) | 2 (20.0) | 0 | 0 | 4 (5.0) | 0 |
| respiratory | | | | | | | | | | |
| tract infection | | | | | | | | | | |
| Various | 0 | 0 | 1 (10.0) | 0 | 0 | 0 | 0 | 0 | 1 (1.3) | 0 |
| neurological | | | | | | | | | | |
| diseases | | | | | | | | | | |
| Dizziness | 0 | 0 | 1 (10.0) | 0 | 0 | 0 | 0 | 0 | 1 (1.3) | 0 |
| Gastrointestinal | 1 (10.0) | 0 | 0 | 0 | 0 | 0 | 0 | 0 | 1 (1.3) | 0 |
| diseases | | | | | | | | | | |
| Indigestion | 1 (10.0) | 0 | 0 | 0 | 0 | 0 | 0 | 0 | 1 (1.3) | 0 |
| Heart disease | 0 | 0 | 0 | 1 (10.0) | 0 | 2 (20.0) | 0 | 0 | 3 (3.8) | 1 (6.3) |
| Sinus | 0 | 0 | 0 | 1 (10.0) | 0 | 2 (20.0) | 0 | 0 | 3 (3.8) | 1 (6.3) |
| bradycardia | | | | | | | | | | |

Note:
Adverse events were coded according to MedDRA V23.0.

In summary, the safety, tolerability, pharmacokinetic, and pharmacodynamic characteristics of a single oral administration of compound (I) capsules to healthy adult subjects were studied in this example. The trial results showed that for compound (I), within a single administration dose range between 10 mg and 800 mg, Tₘₐₓ ranged from 1.03 h to 2.00 h, T_{1/2} ranged from 0.847 h to 8.67 h, and Cₘₐₓ ranged from 46.1 ng/mL to 5060 ng/mL, which increased with increasing dose, showing a linear pharmacokinetic trend. AUC₀₋ₜ ranged from 77.7 h*ng/m to 14392 h*ng/m, which increased with increasing dose within the dose ranges between 10 mg and 100 mg and between 200 mg and 800 mg, showing a linear pharmacokinetic trend. CL/F was similar in the low-dose group (10 mg to 50 mg), and decreased with increasing dose. MRT was short, ranging from 1.53 h to 4.25 h, and was slightly prolonged with increasing dose. The maximum BTK occupancy rate in the 10 mg dose group was 58.93%, and the maximum BTK occupancy rate in the 25 mg and above dose groups was close to 100%. All AEs in the trial group were grade 1 in severity, and only one grade 2 AE occurred in the placebo group.

A Phase IIa clinical trial was designed to evaluate the efficacy and safety of compound (I) after 4 weeks of administration, with a primary efficacy analysis conducted in the fourth week. The Phase IIa clinical trial adopted a randomized, double-blind, placebo-controlled design to evaluate the efficacy and safety of compound (I) in patients. The trial enrolled patients who had been diagnosed with chronic spontaneous urticaria (CSU) at the time of randomization and met the following requirements: (1) itch and hives persisted for at least 6 weeks before screening, despite taking second-generation H1 antihistamines for CSU during this period; (2) urticaria activity score over 7 days (UAS7) (range 0-42)≥16 points, hives severity score over 7 days (HSS7) (range 0-21)≥6 points, and itch severity score over 7 days (ISS7) (range 0-21)≥6 points for 7 consecutive days (−8 to −2 days) before randomization; (3) CSU duration ≥6 months before screening (defined as CSU onset confirmed by the investigator based on all available supporting records). Patients were randomized to receive compound (I) or placebo at a dose of 25 mg twice daily. The preliminary results were evaluated by using the UAS7, itch severity score over 7 days (ISS7), hives severity score over 7 days (HSS7), dermatology life quality index (DLQI), urticaria control test (UCT), and angioedema activity score (AAS7) scales to assess the improvement of patients' urticaria-related symptoms in the 4th or 5th week after administration.

The Phase IIa trial would evaluate the ability of compound (I) to improve urticaria-related symptoms in patients with chronic spontaneous urticaria, as well as its safety in patients.

In the specification, the description of the reference terms such as “one embodiment”, “some embodiments”, “example”, “specific example”, or “some examples” means that the specific features, structures, materials, or characteristics described with reference to the embodiment or example are included in at least an embodiment or example of the present disclosure. In this specification, exemplary descriptions of the foregoing terms do not necessarily refer to the same embodiment or example. Moreover, the specific features, structures, materials, or characteristics described may be combined in any one or more embodiments or examples in a suitable manner. Furthermore, those skilled in the art may combine different embodiments or examples and features of different embodiments or examples described in this specification, unless they are contradictory to each other.

Although embodiments of the present disclosure are illustrated and described above, it can be understood that the above embodiments are illustrative and should not be construed as limitations of the present disclosure. Those skilled in the art can make changes, modifications, substitutions, and variations to the above embodiments within the scope of the present disclosure.

What is claimed is:

1. A method for treating and/or preventing an immune system disease, the method comprising:
administering to a subject a pharmaceutically acceptable amount of a compound represented by Formula (I), or a stereoisomer, tautomer, solvate or pharmaceutically acceptable salt of the compound represented by Formula (I):

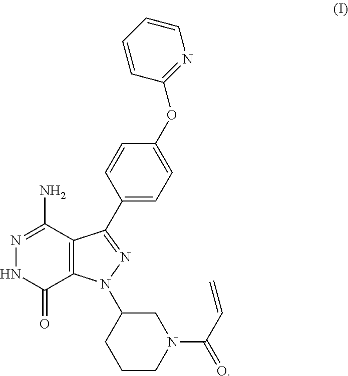 [SMILES: C=CC(=O)N1CCCC(n2nc(-c3ccc(Oc4ccccn4)cc3)c3c(N)n[nH]c(=O)c32)C1]

2. The method according to claim 1, wherein the immune system disease comprises an autoimmune disease and/or a skin allergy disorder.

3. The method according to claim 1, wherein the immune system disease comprises rheumatoid arthritis, urticaria, skin inflammation, and/or eczema.

4. The method according to claim 1, wherein the pharmaceutically acceptable amount of the compound represented by Formula (I), or the stereoisomer, tautomer, solvate or pharmaceutically acceptable salt of the compound represented by Formula (I) ranges from about 5 mg to 800 mg.

5. The method according to claim 1, wherein the pharmaceutically acceptable amount of the compound represented by Formula (I), or the stereoisomer, tautomer, solvate or pharmaceutically acceptable salt of the compound represented by Formula (I) ranges from about 10 mg to 800 mg.

6. The method according to claim 1, wherein the pharmaceutically acceptable amount of the compound represented by Formula (I), or the stereoisomer, tautomer, solvate or pharmaceutically acceptable salt of the compound represented by Formula (I) ranges from about 20 mg to 800 mg.

7. The method according to claim 1, wherein the pharmaceutically acceptable amount of the compound represented by Formula (I), or the stereoisomer, tautomer, solvate or pharmaceutically acceptable salt of the compound represented by Formula (I) ranges from about 25 mg to 800 mg.

8. A single-dose medicament, comprising, as an active ingredient, about 1 mg to 800 mg of a compound represented by Formula (I), or a stereoisomer, tautomer, solvate or pharmaceutically acceptable salt of the compound represented by Formula (I):

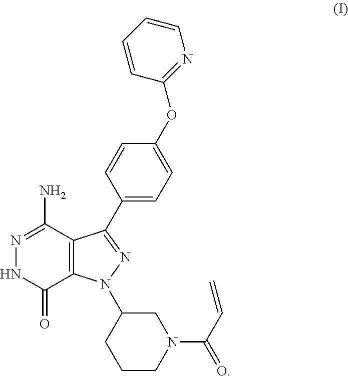 [SMILES: C=CC(=O)N1CCCC(n2nc(-c3ccc(Oc4ccccn4)cc3)c3c(N)n[nH]c(=O)c32)C1]

9. The single-dose medicament according to claim 8, wherein the single-dose medicament is used to treat and/or prevent an immune system disease.

10. The single-dose medicament according to claim 9, wherein the immune system disease comprises an autoimmune disease and/or a skin allergy disorder.

11. The single-dose medicament according to claim 9, wherein the immune system disease comprises rheumatoid arthritis, urticaria, skin inflammation, and/or eczema.

12. The single-dose medicament according to claim 8, comprising, as the active ingredient, about 5 mg to 800 mg of the compound represented by Formula (I), or the stereoisomer, tautomer, solvate or pharmaceutically acceptable salt of the compound represented by Formula (I).

13. The single-dose medicament according to claim 8, comprising, as the active ingredient, about 10 mg to 800 mg of the compound represented by Formula (I), or the stereoisomer, tautomer, solvate or pharmaceutically acceptable salt of the compound represented by Formula (I).

14. The single-dose medicament according to claim 8, comprising, as the active ingredient, about 20 mg to 800 mg of the compound represented by Formula (I), or the stereoisomer, tautomer, solvate or pharmaceutically acceptable salt of the compound represented by Formula (I).

15. The single-dose medicament according to claim 8, comprising, as the active ingredient, about 25 mg to 800 mg of the compound represented by Formula (I), or the stereoisomer, tautomer, solvate or pharmaceutically acceptable salt of the compound represented by Formula (I).

16. A method for treating and/or preventing an immune system disease, the method comprising:
administering to a subject a pharmaceutically acceptable dose of the single-dose medicament according to claim 8.

17. The method according to claim 16, wherein the immune system disease comprises an autoimmune disease and/or a skin allergy disorder.

18. The method according to claim 16, wherein the immune system disease comprises rheumatoid arthritis, urticaria, skin inflammation, and/or eczema.
